(19) United States
(12) Patent Application Publication
(10) Pub. No.: US 2013/0034901 A1
(43) Pub. Date: Feb. 7, 2013

(54) Use of a Proteolytic Enzyme for the Modification of the Cell Surface of a Stem Cell

(71) Applicants: Johanna Nystedt, Helsinki (FI); Tanja Hakkarainen, Helsinki (FI); Petri Lehenkari, Oulu (FI); Erja Kerkelä, Helsinki (FI); Leena Valmu, Helsinki (FI)

(72) Inventors: Johanna Nystedt, Helsinki (FI); Tanja Hakkarainen, Helsinki (FI); Petri Lehenkari, Oulu (FI); Erja Kerkelä, Helsinki (FI); Leena Valmu, Helsinki (FI)

(73) Assignee: SUOMEN PUNAINEN RISTI VERIPALVELU, Helsinki (FI)

(21) Appl. No.: 13/639,604

(22) PCT Filed: Apr. 6, 2011
(86) PCT No.: PCT/FI11/50298

§ 371 Date: Oct. 5, 2012

(30) Foreign Application Priority Data
Apr. 6, 2010 (FI) 20105348

(51) Int. Cl. C12N 5/0789

(52) U.S. Cl. 435/372; 435/375

(57) ABSTRACT

The present invention relates to a stem cell and/or a population thereof having a specific profile of cell surface proteins and/or proteoglycans. The present invention also relates to use of a proteolytic enzyme in the modification of the cell surface of a stem cell. The present invention further relates to a method of modifying the cell surface of a stem cell by treatment with a proteolytic enzyme.

### FIELD OF THE INVENTION

The present invention relates to a stem cell and/or a population thereof having a specific profile of cell surface proteins and/or proteoglycans. The present invention also relates to use of a proteolytic enzyme in the modification of the cell surface of a stem cell. The present invention further relates to a method of modifying the cell surface of a stem cell by treatment with a proteolytic enzyme.

### BACKGROUND OF THE INVENTION

Proteolytic enzymes are a large group of enzymes which are involved in digesting protein chains into shorter fragments by splitting the peptide bonds that link amino acid residues together. Some of them are able to detach the terminal amino acids from the peptide or protein chain (exopeptidases, such as aminopeptidases) and the others attack internal peptide bonds of a protein (endopeptidases, such as trypsin, chymotrypsin, pepsin, papain, elastase). Proteolytic enzymes can be divided into four major groups according to the character of their catalytic active site and conditions of action: serine proteinases, cysteine (thiol) proteinases, aspartic proteinases, and metalloproteinases. Classification of a protease to a certain group depends on the structure of catalytic site and the amino acid (as one of the constituents) essential for its activity.

Pronase is a non-specific, commercially available mixture of proteinases isolated from the extracellular fluid of Streptomyces griseus. Its proteolytic activity is attributable to the composition of the preparation, which comprises various types of endopeptidase (serine and metalloproteases) and exopeptidase (carboxypeptidases and aminopeptidases). Typically, neutral protease, chymotrypsin, trypsin, carboxypeptidase, and aminopeptidase are present, together with neutral and alkaline phosphatases. The preparation is, however, free from nucleases.

Other proteolytic enzymes comprise, e.g. trypsin (EC 3.4.21.4), papain (EC 3.4.22.2), elastase (EC 3.4.21.36), subtilisin (EC 3.4.21.62), proteinase K (EC 3.4.21.64) and kallikrein (EC 3.4.21.34).

Stem cells are characterized by their ability to renew themselves through mitotic cell division and to differentiate into a diverse range of cell types. The two main types of mammalian stem cells are embryonic stem cells and adult stem cells, such as hematopoietic stem cells, mesenchymal stem cells, endothelial stem cells and tissue-specific stem cells. Induced pluripotent stem (iPS) cells are derived from adult tissues but converted to embryonic stem cell like cells.

Hematopoietic stem cells (HSC) form progenitors for practically all cell types found in the blood. HSC are currently used for treating many malignant hematological diseases, in particular leukemias and also certain nonhematological diseases. HSCs can be found in and typically are isolated from, for example, bone marrow and cord blood. Usually, HSC are selected using CD34 or CD133 as markers; but similar to other stem cells there are no definitive cell surface markers for HSC (e.g. Spangrude, Uchida and Weissman: Hematopoietic stem cells: biological targets and therapeutic tools. In Atkinson et al, eds: Clinical Bone Marrow and Blood Stem Cell Transplantation, pp 13-37. Cambridge Univ Press, Cambridge U.K., 3^{rd} ed, 2004).

Mesenchymal stem cells (often also called as mesenchymal stromal cells; MSC) have the potential to differentiate into various cellular lineages and can be expanded in culture conditions without losing their multipotency. Cell types that MSCs have been shown to differentiate into in vitro and/or in vivo include osteoblasts, chondrocytes and adipocytes. Therefore, they present a valuable source for applications in cell therapy and tissue engineering. MSCs can be derived, for example, from bone marrow or cord blood. The exact definitions for MSC or cell lineages differentiated thereof are currently not finally established (Da Silva Meilleres et al., Stem Cells 2008; 26: 2287-99), but an example of a current set of criteria for undifferentiated MSC is described by Dominici et al., in Cytotherapy 2006; 8: 315-317, but the markers do not detect a single homogeneous population. Hence, MSC as defined currently is a heterogeneous cell population. Transplantation of MSC offers a promising approach for treating certain nonhematological malignant and nonmalignant diseases and for stem cell-mediated tissue regeneration. In particular, they can be applied to induce immunosuppression (Nauta and Fibbe, Immunomudulatory properties of mesenchymal stromal cells. Blood 2007; 110: 3499-3506). This can be done as supportive therapy in hematological stem cell transplantation in which immunologically-mediated graft-versus-host disease is a major complication. Immunomodulation also has a great potential in autoimmune or immune-mediated diseases, such as multiple sclerosis, rheumatoid artritis, or inflammatory bowel disease. In addition to the immunomodulation, MSC can be therapeutically used, for example, to induce angiogenesis or to produce collagen, a therapeutic possibility in rheumatoid arthritis.

In addition to hematopoietic and mesenchymal stem cells, the present invention can be used with induced pluripotent stem (iPS) cells. iPS cells are a type of pluripotent stem cell derived or produced from principally any adult non-pluripotent or differentiated cell type, such as an adult somatic cell, that has been induced to have all essential features of embryonic stem cells (ESC). The techniques were first described in human cells by Takahashi et al. in Cell 131: 861-872, 2007. There are currently a number of ways to make iPS cells. Their therapeutic potential has been predicted to be enormous because patient's own cells can be induced and hence, ethical and histocompatibility problems can be avoided.

Embryonic stem cells (ESC) are pluripotent stem cells derived from the inner cell mass of the blastocyst, an early-stage embryo. Cells derived from ESC are also developed for therapeutic purposes, for example, Jiang et al in Stem Cells 2007; 25: 1940-1953. In technologies for harvesting hESCs, the embryo is either destroyed or not, i.e. it remains alive. In one embodiment of the invention, the hESCs are harvested solely by a method that does not include the destruction of a human embryo.

A problem related to stem cell transplantation as done using current standards is entrapment (may also be called as “distribution” or “homing”) of the transplanted cells to unwanted organs or tissues. The term “homing” here refers to targeted trafficking of cells to certain tissues or organs; often mediated by specific cell surface molecules and soluble chemokines. One example of undesired distribution of cells is the observed phenomena of lung entrapment: intravenously infused MSCs are rapidly trapped in the lungs in animal models (Gao et al. 2001. The dynamic in vivo distribution of bone marrow-derived mesenchymal stem cells after infusion. Cells Tissues Organs 169, 12-20; Schrepfer et al. 2007. Stem cell transplantation: the lung barrier. Transplant Proc 39: 573-576). Lung entrapment is not limited to MSCs, since trapping of cells in the lungs occurs also with metastatic tumors (Khanna et al 2004. The membrane-cytoskeleton linker ezrin is necessary for osteosarcoma metastasis. Nature Med 10, 182-186) and with HSCs during acute distribution (Kang et al. 2006: Tissue distribution of 18F-FDG-labeled peripheral hematopoietic stem cells after intracoronary administration in patients with myocardial infarction. J Nucl Med. 47:1295-301).

An approach to increase the portion of the cells that find their way to the intended target organ or tissue, has been increasing the number of cells in a graft. However, by this approach the increased dose unfortunately tends to result in higher rates of clinical complications, for example, graft-versus-host disease, a life threatening condition after HSC transplantation. Also, it is sometimes not feasible to get a higher number of cells for transplantation, for example, a single unit of cord blood, a suitable source for stem cells, has a limited number of stem cells. Expansion ex vivo provides one option to get a higher number of stem cells but it is currently not established that the expanded cells have the same properties as the original cells. Hence, a more efficient use of stem cells of a graft is warranted.

It has now been discovered that by processing and/or treating stem cells with a proteolytic enzyme, the relative amount of cells entrapped to the lungs and liver diminishes after transplantation and/or the relative amount of cells finding their way to the desired target organs increases.

### BRIEF DESCRIPTION OF THE INVENTION

An object of the present invention is to provide a stem cell and/or a population thereof, having a specific profile of cell surface proteins and/or proteolglycans. Another object of the present invention is to provide a method of modifying the cell surface of a stem cell by treatment with a proteolytic enzyme. Another object of the invention is a use of a proteolytic enzyme in the modification of the cell surface of a therapeutic cell preparation.

In particular, an object of the present invention is to provide a method of assisting therapeutic stem cells to the target organ(s) or tissues. Another object of the present invention is to provide a method of hindering and/or preventing the transition of stem cells from blood stream to organs which are not the actual target ones, e.g., the lungs and/or liver. A further object of the present invention is to provide a method of modifying and/or altering the distribution behaviour of cells used for cellular therapy.

The invention is based on the observation that stem cells treated with pronase are entrapped to a lesser extent to the lungs, i.e., to organs which are not the actual targets of the stem cell graft, than stem cells that have not been treated with pronase or have been treated with trypsin, another proteolytic enzyme.

Accordingly, the present invention provides a novel and effective means for assisting the transition of stem cells of a graft, and other therapeutic stem cells from blood stream to the target organ(s) and optionally simultaneously hindering and/or preventing the transition of stem cells of a graft from blood to organs which are not the actual targets, i.e., the lungs and/or liver. In addition, the present invention provides a novel and effective means for modifying and/or altering the “homing” properties or behaviour of the cells.

The objects of the invention are achieved by the methods, uses and cell populations set forth in the independent claims. Preferred embodiments of the invention are described in the dependent claims.

Other objects, details and advantages of the present invention will become apparent from the following drawings, detailed description and examples.

### BRIEF DESCRIPTION OF THE DRAWINGS

FIG. 1 shows the fibronectin cell surface expression in A. MSCs and B. HSCs, as determined by flow cytometry. In the example shown, fibronectin positive cells are encircled, and CD34+ HSCs double positive cells for fibronectin is encircled in B. All CD34+ cells are fibronectin positive, since no positive cells remain in quadrant 4 (Q4).

FIG. 2 shows that the cell surface fibronectin expression increases with confluency of cultured cells and in an enforced suspension incubation of adherent cells. Flow cytometric analysis A. MSC fibronectin expression during different levels of confluency. Left panel shows results as percentage (%) of fibronectin positive cells, right panel as intensity of fluorescence. B. Fibronectin expression during enforced suspension incubation of MSCs. Both confluency and suspension incubation increases cell surface fibronectin expression of MSCs.

FIG. 3 shows the mitochondrial inner potential of MSCs as measured with the JC-1 label and flow cytometry after trypsin or 0.5% pronase detachment. There are less late apoptotic and necrotic cells after pronase detachment.

FIG. 4 shows the cell surface fibronectin expression of MSCs after A. 30 min suspension incubations with different concentrations of pronase and B. detachment from culture vessel with different concentrations of pronase. Flow cytometric analysis, results presented as percentage of anti-fibronectin antibody stained positive cells.

FIG. 5 shows the cell surface antigen expression of MSCs as determined by flow cytometry after either trypsin or pronase (0.5%) detachment.

FIG. 6 shows the efficiency and stability of pronase or trypsin MSC detachment as studied with suspension incubation (recovery) after detachment for 90 and 180 min. PRN=pronase, TrypLE=trypsin.

FIG. 7 shows osteogenic and adipogenic differentiation capacity of pronase detached cells. Representative phase contrast pictures. Osteogenic differentiation visualized by von Kossa staining, black staining indicates level of mineralization. Adipogenic differentiation by Sudan III staining, red staining indicates fat droplets.

FIG. 8 shows that pronase treatment decreases the pulmonary trapping of UCBMSCs. A) Radioactivity of the lungs and B) Gamma camera images 1 hour after UCBMSC administration. C) Radioactivity of the lungs and D) Gamma camera images 15 hours after UCBMSC administration. E-G) Radio-activity of the lungs in comparison to liver, GI tract and bone marrow 15 hours after UCBMSC administration. Number of PHK-labelled UCB MSCs detected from lungs, spleen, bone marrow, and peripheral blood 1 hour (H and I) and 20 hours (J and K) post injection. n=3 in radioactive experiments, n=4 in PKH-26 experiment. cpm;counts per minute, CTRL;trypsin treated cells, Modified; pronase treated cells, BM; Bone marrow.

FIG. 9 shows that pronase treatment decreases the pulmonary trapping of BMMSCs. A) Radioactivity of the lungs and B) Gamma camera images 1 hour after BMMSC administration. C) Radioactivity of the lungs and D) Gamma camera images 15 hours after BMMSC administration. E-F) Radioactivity of the lungs in comparison to liver and GI tract 15 hours after BMMSC administration. n=5 in all experiments. cpm; counts per minute, CTRL;trypsin treated cells, Modified; pronase treated cells.

FIG. 10 shows the cell surface profiling by FACS described in Example 5.

FIG. 11 A) shows the proteins of UCB MSC digested more effectively by pronase than trypsin as determined by mass spectrometry; B) shows examples of the proteins digested by pronase verified as reduced antibody staining in flow cytometry. In 11A, UniProtKB Database identifiers and protein names are given as well as the number of peptides detected after the enzymatic treatments and the number of peptide fragments disappearing after the pronase treatment. The last column shows the verification of mass spectrometric results by antibody stainings after pronase and trypsin treatments.

FIG. 12 shows the effect of A) subtilisin, B) proteinase K and C) elastase on the cell surface as studied by flow cytometry. %-positive cells of surface proteins after different enzymatic treatments with two concentrations are shown; the trypsin treated cells were used as control. Two BM MSC lines 437 and 428 were used.

FIG. 13 shows the FACS results as %-positive cells of selected surface proteins after different enzymatic treatments in UCB MSC line 391P. The cells were treated with A) subtilisin, B) proteinase K, and C) elastase. Trypsin treated cells were used as control.

FIG. 14 shows the immunosuppressive effect of the UCB MSCs after pronase and trypsin treatment. Fluorescence-labelled mononuclear cells (MNC) from two individuals, BCL23 and BCL24, were tested. T-cell proliferation was activated with a CD3 antibody (clone Hit3a). Trypsinized or pronase treated MSCs, (A) line 391P (p4) and (B) line 588P (p5) were co-cultured with MNCs for four days after which the MNCs were analyzed with flow cytometry. Immunosuppressive effect was measured as the ability of MSCs to inhibit the proliferation of T-cells. The immunosuppressive ability of the pronase-treated cells was comparable to that of control cells.

FIG. 15 shows the angiogenic effect of A) BMMSC and B) UCBMSC in angiogenesis co-culture assay. The angiogenic ability of the pronase-treated cells (right column) was comparable to that of control cells (middle column). The medium alone induced A) no angiogenesis, or B) only a low level of angiogenesis. Different treatments were compared to their respective controls and the results are shown as % of tubule formation compared to positive control. The results were significant when p<0.05*, p<0.01**, p<0.001*** as tested with ANOVA. BM medium=bone marrow stem cell medium, UCB medium=umbilical cord blood stem cell medium, Ctrl=control cells, Pronase=pronase-treated cells.

FIG. 16 demonstrates the results of in vivo studies in an experimental porcine model. A) shows that the relative amount of radioactivity in the lungs was lower in the pronase-treated group (right bar in each column) than in the control trypsin-treated group (left bar in each column) (n=2 animals per group; the means are shown). Pulm I.dx and I.sin=the right and left lungs; Ren I.dx and I.sin=kidneys, respectively. B) Shows the reduced expression of CD44 and fibronectin on cell surface after pronase treatment compared to trypsin-treatment in two porcine Bone Marrow MSC cell lines (I and II).

FIG. 17 lists the antibodies used in the invention.

### DETAILED DESCRIPTION OF THE INVENTION

It has been shown in the present invention that fibronectin can be detected on the surface of MSC and HSC. Also, the expression of the cell surface fibronectin increases along with the confluence of adherent cultured cells and with enforced suspension incubation of adherent cells.

It has also been shown in the present invention that pronase removes or cleaves off the cell surface fibronectin on MCSs. The treatment can be done in cell suspension incubation or when detaching adherent cells from culture dishes. In both cases the cells remain alive and keep their stem cell-related functions, such as multipotency or immunosuppressive ability. Further, in addition to the effective cleavage of fibronectin from the cell surface, pronase produces other changes in the protein profile of the cell surface as demonstrated by the disappearance of certain antibody-binding epitopes. In particular, the binding of the anti-CD44 antibody is practically completely vanished, the staining being about 1-2% or practically 0% depending on the conditions used, indicating a cleavage of the hyaluronan receptor CD44 (UniProt id P16070). Cleavage is also seen for the epitope of anti-CD105 antibody which showed staining of about 60-80% with a low concentration of pronase and only 0-2% with a higher pronase concentration. CD105 is also called endoglin and the human CD105 has UniProt id P17813. The integrins CD49d (α4; UniProt id P13612) and CD49e (α5; UniProt id P08648) showed similar patterns as CD105. More than 90% of untreated cells stained positive for these antigens but the staining was diminished after the pronase treatment. Galectin-1 (Uni-Prot id P09382), CD166 (ALCAM, UniProt id Q13740), CD146 (MUC18, Uni-Prot id P43121) and chondroitin sulfate proteoglycan 4 (CSPG4, UniProt id Q6UVK1) were also diminished after the pronase treatment. The decrease in the expression levels of CD44, CD105, CD49e, galectin-1, CD166, CD146 and CSPG4 proteins could also be demonstrated by mass spectrometric analysis. On the other hand, antibody epitopes for proteins CD90 (Thy-1, UniProt id P04216), CD29 (integrin beta-1; UniProt id P05556) and/or CD13 (aminopeptidase N; EC 3.4.11.2; UniProt id P15144) remain practically intact after the pronase treatment. The effects of pronase treatment, hence, were specific and dose-dependent and produced a cell type with unique profile or composition of cell surface proteins. In addition, proteinase K treatment was found to produce similar changes than pronase on the cell surface. Specifically, proteinase K treatment was found to diminish the amount of fibronectin and CD44 on the cell surface.

Thus in one embodiment, the present invention provides a stem cell and/or a population thereof having cell surface protein profile wherein proteins fibronectin and CD44 are “essentially missing” or their level is less than 10%, preferably less than 5% of those detected in untreated or control (e.g. trypsin-treated) cells or generally detected in cells that contain these proteins on their surface. In another embodiment of the invention, the level of at least one of the proteins CD49d, CD49e, CD105, galectin-1, CD166, CD146 and/or CSPG4 is additionally “diminished” to less than 70%, preferably less than 40%, of the levels observed in untreated cells or generally detected in cells that contain these proteins on their surface. In a further embodiment, the present invention provides a stem cell and/or a population thereof having cell surface protein profile wherein in addition to proteins fibronectin and CD44 also at least one of proteins CD49d, CD49e CD105, galectin-1, CD166, CD146, and/or CSPG4 is “essentially missing”.

Furthermore in one embodiment, the present invention provides a stem cell and/or a population thereof having the cell surface protein profile wherein proteins fibronectin and CD44 are “essentially missing” and proteins CD90, CD29 and CD13 are “present” in the profile. In a further embodiment, the present invention provides a stem cell and/or a population thereof having the cell surface protein profile, wherein

(i) proteins fibronectin and CD44 are “essentially missing”, and/or

(ii) at least one of proteins CD49d, CD49e, CD105, galectin-1, CD166, CD146, or CSPG4 is diminished, and/or

(iii) proteins CD90, CD29 and CD13 are “present” in the profile.

In an even further embodiment, the present invention provides a stem cell and/or a population thereof having the cell surface protein profile wherein in addition to proteins fibronectin and CD44, also at least one of proteins CD49d, CD49e, CD105, galectin-1, CD166, CD146 or CSPG4 is “essentially missing”, and proteins CD90, CD29 and CD13 are “present” in the profile.

In one embodiment of the invention, the term “at least one of proteins CD49d, CD49e, CD105, galectin-1, CD166, CD146, or CSPG4” refers to one of proteins CD49d, CD49e, CD105, galectin-1, CD166, CD146, or CSPG4. In another embodiment of the invention, the term refers to any combination of three of proteins CD49d, CD49e, CD105, galectin-1, CD166, CD146, or CSPG4, for example proteins CD49d, CD49e and CD105. In a further embodiment of the invention, the term refers to at least three of proteins CD49d, CD49e, CD105, galectin-1, CD166, CD146, or CSPG4. In an even further embodiment of the invention, the term refers to all of the proteins CD49d, CD49e, CD105, galectin-1, CD166, CD146, and CSPG4.

A stem cell and/or a population thereof having one of the above characterized cell surface protein profiles can be produced by treating the cell or the population thereof with proteolytic enzyme or by preventing the expression of genes coding these molecules by a specific inhibitor and/or by any suitable gene technological means.

Here the term “essentially missing” refers to a level less than 10%, preferably less than 5% of those detected in untreated or control cells or generally detected in cells that contain these proteins in their surface. Term “diminished” here refers to a level of less than 70%, preferably less than 40%, and even more preferably less than 10% of the levels found in untreated or control cells or generally detected in cells that contain these proteins in their surface. Term “present” in the profiles refers to the essentially equal levels to those found in untreated or control cells or generally detected in cells that contain these proteins in their surface; preferably more than 80%, or more preferably more than 90% of the cells being positive. The detection can be done in various methods known in the art, for example, it can be based on antibody epitopes or mass spectrometric analysis. Further, it is generally known that depending on the exact conditions of enzymatic treatment e.g. time, concentration of the enzyme, buffer and/or temperature, the level of decrease of the proteins on the cell surface can vary.

The stem cells and/or the populations thereof having the cell surface protein profile according to the present invention are suitable to be used as a clinical graft for transplantation or in cellular therapy. They are found to a lesser extent to entrap or “home” to organs such as, the lungs and liver, which are not the actual target organs of the stem cell graft.

The term “a stem cell” refers to an adult stem cell, such as a mesenchymal, hematopoietic or endothelial stem cell and/or an embryonal stem cell and/or an iPS cell.

The results indicate that a different cell surface can be produced by pronase treatment and that for many cell surface antigens the effect is transient with a recovery process initiated after a certain time period, such as 90-180 min. It is, however, noteworthy that the effect is more stable for fibronectin, an abundant extra cellular matrix (ECM) protein. Pronase treatment can thus be used as a relevant method to produce cells without cell surface fibronectin and a transiently altered cell surface for certain markers antigens. Additionally, the altered cell surface is transient, implying that the cells gain back their original cell surface profile, apparently with their functional properties as well. The recovery, however, is not too rapid for effective changes in tissue targeting of the cells or for its effective application in the clinical setting. Pronase treatment does not, however, affect in vitro multipotency capacity of the stem cells: Both osteogenic and adipogenic differentiation took place successfully after pronase treatment. Similarly, the cells did not lose their ability to suppress immune activation or their capacity to promote angiogenesis. This implies that the treatment with pronase does not destroy the therapeutic potential of the cells. In addition, it was found in an experimental animal model that pronase treatment decreased the pulmonary trapping of the stem cells.

The treatment of stem cells by proteases, typically trypsin, for detachment is described in prior art but what was surprising in the present invention was the “homing” properties of the treated stem cells. Thus, the invention is based on the finding that stem cells treated with the proteolytic enzyme are to a lesser extent entrapping or “homing” to the lungs and liver, i.e., to organs which are not the actual targets of the stem cell graft in e.g. typical HSC or MSC transplantation, than stem cells that have not been treated or have been treated with trypsin.

On the basis of this finding, a method of modifying the cell surface of a stem cell by treatment with a proteolytic enzyme has been developed. The stem cell and/or a population thereof treated with a proteolytic enzyme have a unique cell surface protein and/or proteoglycan profile. Further, a smaller number of the pronase-treated cells find their way to the organs which are not their actual targets.

Accordingly, the present invention provides a novel and effective means for assisting the transition of transplanted stem cells to the target organ(s) and optionally simultaneously hindering and/or preventing the transition of the cells from the blood to organs which are not their actual targets, i.e., the lungs and/or liver. In addition, the present invention provides a novel and effective means for modifying and/or altering the distribution properties and behaviour of therapeutic cells.

The stem cells treated with pronase remain stem cell-like and maintain the characteristics typical and peculiar to stem cells.

In the present invention, the term “proteolytic enzyme” refers to pronase and “pronase-type enzymes” and to proteinase K. In the present invention, the term “pronase-type enzyme” refers to a proteolytic enzyme or a mixture of enzymes that cleavages proteins and/or peptide chains essentially similarly than pronase or has an essentially similar mixture of enzymes as found in typical pronase preparations. In the present invention, the term “proteolytic enzyme” does not refer to trypsin. In a preferred embodiment of the present invention, the proteolytic enzyme is pronase. In another embodiment of the present invention, the proteolytic enzyme is pronase-type enzyme. In a further embodiment, the enzyme is proteinase K used alone or together with pronase.

Pronase treatment can optionally be combined with other treatments and/or modification, such as trypsin treatment before the pronase treatment. Further, the pronase treatment can be combined with other suitable treatments, such as enzymatic modification of glycan structures of glycoproteins. Examples of this are addition of fucose by fucosyl transferases, or addition or removal of sialic acid residues (WO 2008 087256; Xia et al. 2004. Surface fucosylation of human cord blood cells augments binding to P-selectin and E-selectin and enhances engraftment in bone marrow. Blood 104:3091-3096; Sackstein et al. 2008. Ex vivo glycan engineering of CD44 programs human multipotent mesenchymal stromal cell trafficking to bone. Nature Med 14:181-187).

Accordingly, the present invention relates to use of a proteolytic enzyme for the modification of the cell surface of a therapeutic cell. The present invention relates also to a method of modifying the cell surface of a cell by treating the cell with a proteolytic enzyme. In one embodiment of the invention, the cell is a mesenchymal stem cell and/or a population thereof, or a hematopoietic stem cell and/or a population thereof. The method may also contain additional and/or optional steps that are conventional to methods of modifying cells, such as washing, incubating and dividing the cell populations.

In a typical embodiment, merely to give an illustration of the methodology that can be applied in the invention, adherent MSC are cultivated in plastic cell culture vessels with e.g. Ø 10-15 cm, until an optimal passage number or population doubling is reached (e.g. passage 2-3 after establishing the primary culture). When reaching optimal confluency for cell harvesting, the culture medium is removed and the cells are washed once with e.g. PBS w/Ca and Mg ions, pH 7.2 with 0.5 mM EDTA. After removing the wash buffer, the cells are detached with 0.05 ml/cm² prewarmed (+37° C.) 0.05-1% (w/vol) pronase in PBS w/Ca and Mg pH 7.2 with 0.5 mM EDTA buffer until detached. The detachment usually takes place between 3-7 minutes at +37° C. The pronase detachment is subsequently stopped by adding excess cell culture media containing serum. The detached cells can be collected at this stage. After pelleting by centrifugation, the cells can be dissolved in any buffer suitable for injection. HSC that are non-adherent can be selected and isolated from different biological materials by utilizing suitable cell surface antigens, such as CD34. Unselected or selected HSCs can subsequently also be in vitro expanded as suspension cultures in optimized media supplemented with serum and relevant cytokines for 5-20 days. Cells in suspension can also be treated with e.g. 0.05-100 μg pronase in expansion media or e.g. αMEM+0.5% (vol/vol) human serum albumin (HSA) with 3-7×10e5 cells/ml for 5-30 min at +37° C., The suspension pronase treatment is stopped by adding excess buffer and pelleting the cells by centrifugation, removing the supernatant and dissolving the cells in any buffer suitable for injection. The cells produced can be injected directly to a patient or stored in liquid nitrogen at −196° C. in optimal storage buffers, also in those supplemented with DMSO.

The present invention additionally relates to a method of assisting the transition of stem cells of a graft to the target organ of an individual by treating the cells with a proteolytic enzyme and injecting them to the individual in the need of such engraftment. Further, the present invention relates to a method of assisting the transition of stem cells of a graft from the blood stream to the target organ of an individual by treating the cells with a proteolytic enzyme and injecting them to the blood stream of an individual in the need of such engraftment. In one embodiment of the invention, the cells are treated with the proteolytic enzyme in vitro.

Furthermore, the present invention relates to a method of hindering and/or preventing the transition of stem cells of a graft from blood stream to an organ that is not the actual target one by treating the cells with a proteolytic enzyme. In one embodiment of the invention, the organ that is not the actual target organ is lungs and/or liver.

The present invention also relates to a method of modifying and/or altering the distribution behaviour of stem cells in a graft with the treatment of a proteolytic enzyme.

The present invention further relates to a stem cell and/or a population thereof having cell surface protein and/or proteoglycan profile resulting from the treatment with a proteolytic enzyme. In one embodiment of the invention, the stem cell and/or a population thereof are a mesenchymal stem cell and/or a population thereof, or a hematopoietic stem cell and/or a population thereof.

It is of note that in addition to the stem cells, many other cells types are known for cellular therapy. Many of them have the kind of problems related to trapping to unwanted tissues. Other cell types include regulatory T lymphocytes and macrophages that are used for immunomodulatory effects, and cytotoxic T lymphocytes and natural killer (NK) cells used for targeted immune response. In all these cases the therapeutic efficiency of the preparation can be augmented by hindering entrapping in the lung or liver. These cells can be applied alone or together with some other cells, such as stem cells.

It will be obvious to a person skilled in the art that, as the technology advances, the inventive concept can be implemented in various ways. The invention and its embodiments are not limited to the examples described above but may vary within the scope of the claims.

The invention will be described in more detail by means of the following examples. The examples are not to be construed to limit the claims in any manner whatsoever.

### EXAMPLE 1

### Materials and Methods

Umbilical cord blood-derived mesenchymal stem cell (UCBMSC) 594P in p2, UCBMSC 454T(7) in p4 and Ficoll-isolated UCB-derived mononuclear cells (MNCs) were used in the experiments. The adherent MSCs were detached in 70-100% confluency with trypsin (TryPLe Express, Invitrogen) and the trypsinization was stopped within 4 minutes with excess culture media. The cells were labelled for flow cytometric analysis with 2 μl of the anti-fibronectin antibody (#ab6327, abcam, FIG. 17) and PE-conjugated anti-CD34 antibody (#130-081-002, Miltenyi Biotec) per 1×10e5 cells in PBS w/Ca & Mg pH 7.2+0.5% bovine serum albumin (BSA) for 30 minutes on ice. After washing with excess labelling buffer, secondary antibody staining was done with Alexa 488-conjugated goat-anti mouse IgG (H+L) diluted 1:500. The labelled cells were run with a FACSAria (BD) flow cytometer and the results were analyzed with the FACSDiva software (BD).

### Results

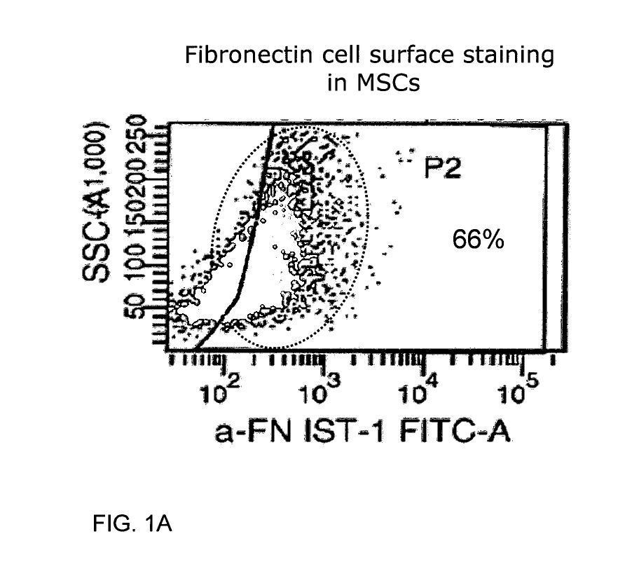

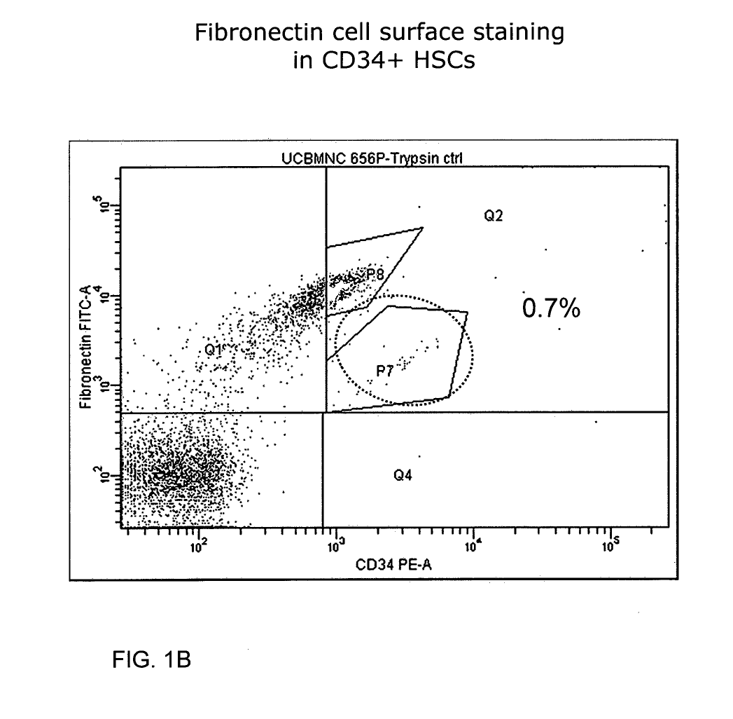

MSCs: The UCBMSCs were concluded to be cell surface fibronectin positive with a varying percentage of 25-60% depending of used MSC line and confluency (see FIG. 1A).

HSCs: Fibronectin staining of CD34+ cells were analyzed by co-labelling Ficoll-isolated UCBMNCs with anti-CD34− and anti-fibronectin-antibodies. The CD34+ cell population of UCBMNCs were concluded to be 100% fibronectin positive (see FIG. 1B).

It is evident that both studied stem cell types (MSC and HSC) express fibronectin on the cell surface and thus are fibronectin positive. The varying degree of MSC fibronectin staining might be explained by alterations in cell surface fibronectin expression due to heterogeneity between the individual MSC lines, passage number and level of cell confluency in culture. Also, the used fibronectin antibody might be unstable after short-term storage. It has been shown previously that HSCs bind to fibronectin (Giancotti et al. 1986).

### EXAMPLE 2

### Materials and Methods

Confluency Experiments:

Umbilical cord blood-derived mesenchymal stem cell (UCBMSC) 454T(7) in p6 were used in the confluency experiments. The cells were plated in different densities to yield different levels of confluency on the same analysis day. The MSCs were analyzed at 40%, 70% and 100% confluency and were simultaneously detached with trypsin (TryPLe Express, Invitrogen). The trypsinization was stopped within 4 minutes with excess culture media. The cells were labelled for flow cytometric analysis with 2 μl of the anti-fibronectin antibody (#ab6327, abcam) per 1×10e5 cells in PBS w/Ca & Mg pH 7.2+0.5% bovine serum albumin (BSA; ultrapure, Sigma) for 30 minutes on ice. After washing with excess labelling buffer, secondary antibody staining was done with Alexa 488-conjugated goat-anti mouse IgG (H+L) diluted 1:500. The labelled cells were run with a FACSAria (BD) flow cytometer and the results were analyzed with the FACSDiva software (BD).

Suspension Incubation Experiments:

Umbilical cord blood-derived mesenchymal stem cell (UCBMSC) 391P in p5 were used in the suspension experiments. The MSCs in subconfluency were detached with trypsin (TryPLe Express, Invitrogen) and the trypsinization was stopped within 4 minutes with excess culture media. The cells were calculated and washed once with PBS w/Ca & Mg pH 7.2+0.5% BSA. Viability was determined by trypan blue exclusion and microscopy and was always concluded to be >99%. The MSCs were subsequently suspension incubated in PBS w/Ca & Mg pH 7.2+0.5% BSA at room temperature at a cell density of 1×10e6 cells/ml for either 90 or 180 min. Viability was also determined after the suspension incubation and remained unchanged even after 180 min. The MSCs were labelled after the indicated suspension incubations for flow cytometric analysis with 2 μl of the anti-fibronectin antibody (#ab6327, abcam) per 1×10e5 cells in PBS w/Ca & Mg pH 7.2+0.5% bovine serum albumin (BSA; ultrapure, Sigma) for 30 minutes on ice. After washing with excess labelling buffer, secondary antibody staining was done with Alexa 488-conjugated goat-anti mouse IgG (H+L) diluted 1:500. The labelled cells were run with a FACSAria (BD) flow cytometer and the results were analyzed with the FACSDiva software (BD).

### Results

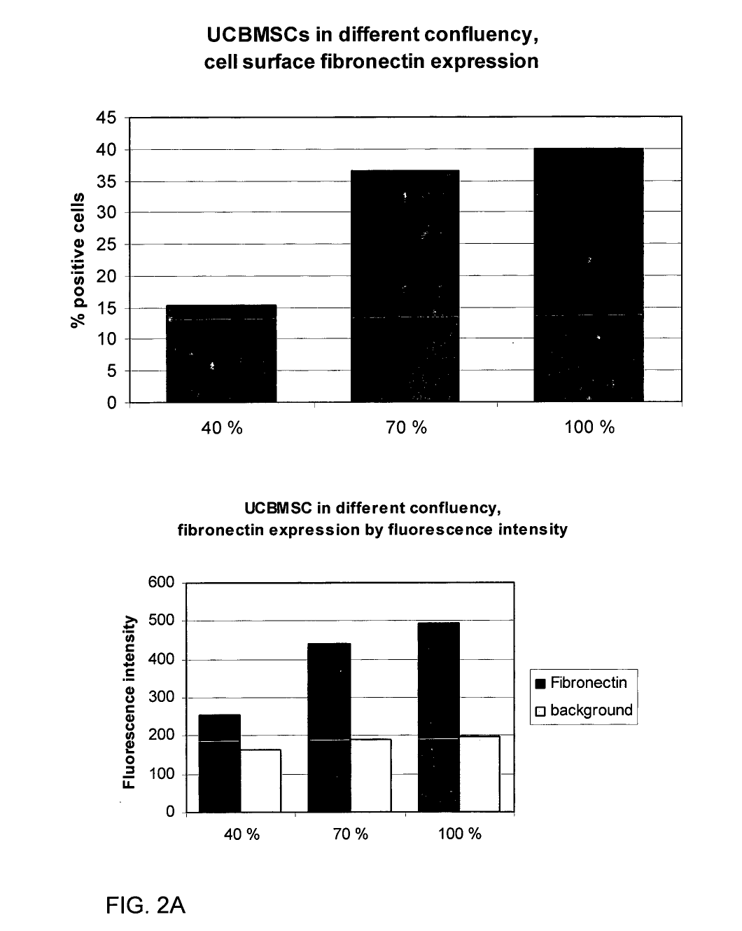

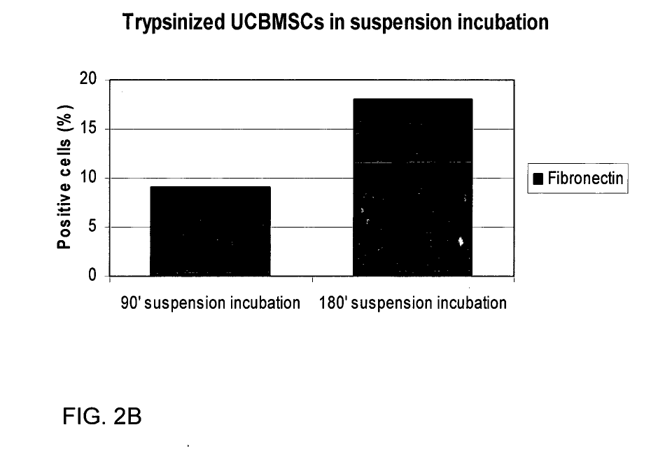

MSC cell surface fibronectin expression increases with both confluence (see FIG. 2A) and with enforced suspension incubation (see FIG. 2B). The results reflect the transient state of a cell surface, where changes in adhesion molecules and extracellular matrix (ECM) proteins occur constantly. Evidently, higher confluency stimulates MSCs to produce more fibronectin. Additionally these results demonstrate that the cell surface of viable adherent cells in suspension, for periods which are relevant to the clinical setting (90-180 min after detachment), also exhibits transient changes and an evident increase in fibronectin expression is seen (see FIG. 2B).

### EXAMPLE 3

### Materials and Methods

Pediatric human bone marrow-derived mesenchymal stem cell (BMMSC) line M2 in passage 6 was used for the study. The subconfluent cells were detached with either trypsin (TryPLE Express, Invitrogen) or 0.5% pronase in PBS-0.5 mM EDTA. Detachment was stopped after 4 minutes by adding excess culture media. Viability was determined by trypan blue exclusion. The mitochondrial inner potential was measured with the JC-1label (Molecular Probes, Invitrogen) and flow cytometry.

### Results

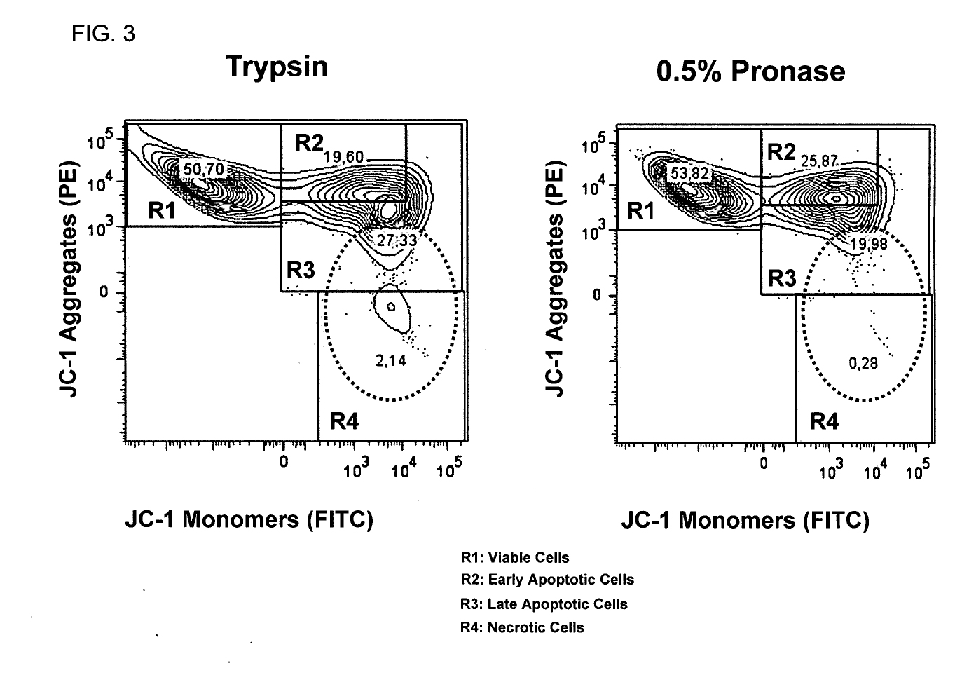

As compared to trypsin, the pronase detachment protocol (maximum concentration tested 0.5% pronase in PBS-EDTA) was as fast as the trypsin detachment protocol. Pronase detachment produced very viable, one-cell MSC suspensions without any cell aggregates. The cells had >95% viability (equal to trypsin) as determined by Trypan blue exclusion. Pronase-detached cells exhibited a better mitochondrial inner potential as studied with the JC-1 label as compared to trypsinized cells (see FIG. 3). As can be seen in FIG. 3, there are less late apoptotic cells (19.98% compared to 27.33% after trypsinization) and necrotic cells (0.28% compared to 2.14% after trypsinization) after 0.5% pronase detachment of BMMSCs.

### EXAMPLE 4

### Materials and Methods

UCBMSC line 454T(7) in passage 4 (p4) was used for these experiments. The subconfluent cells were either:

detached with trypsin (TryPLe Express, Invitrogen) and suspension incubated for 30 min with different concentrations of pronase (#10165921001, Roche) in αMEM Glutamax (Invitrogen)+0.5% human serum albumin (HSA). Buffer incubation without pronase was used as control.

detached by different concentrations of pronase (ranging from 0.05-1%) in PBS w/Ca & Mg pH 7.2+0.5 mM EDTA. Trypsin detachment was used as control.

All samples were done in replicates. Cell viability was determined for all samples after every test by Trypan blue exclusion. Cell morphology was observed for all samples after every test and documented by phase contrast microscopy. The cells were labelled for flow cytometric analysis with 2 μl of the anti-fibronectin antibody #ab6327 (abcam) per 1×10e5 cells in DPBS pH 7.2+0.5% bovine serum albumin (BSA) for 30 minutes on ice. After washing with excess labelling buffer, secondary antibody staining was done with Alexa 488-conjugated goat-anti mouse IgG (H+L) diluted 1:500. The labelled cells were run with a FACSAria (BD) flow cytometer and the results were analyzed with the FACSDiva software (BD).

### Results

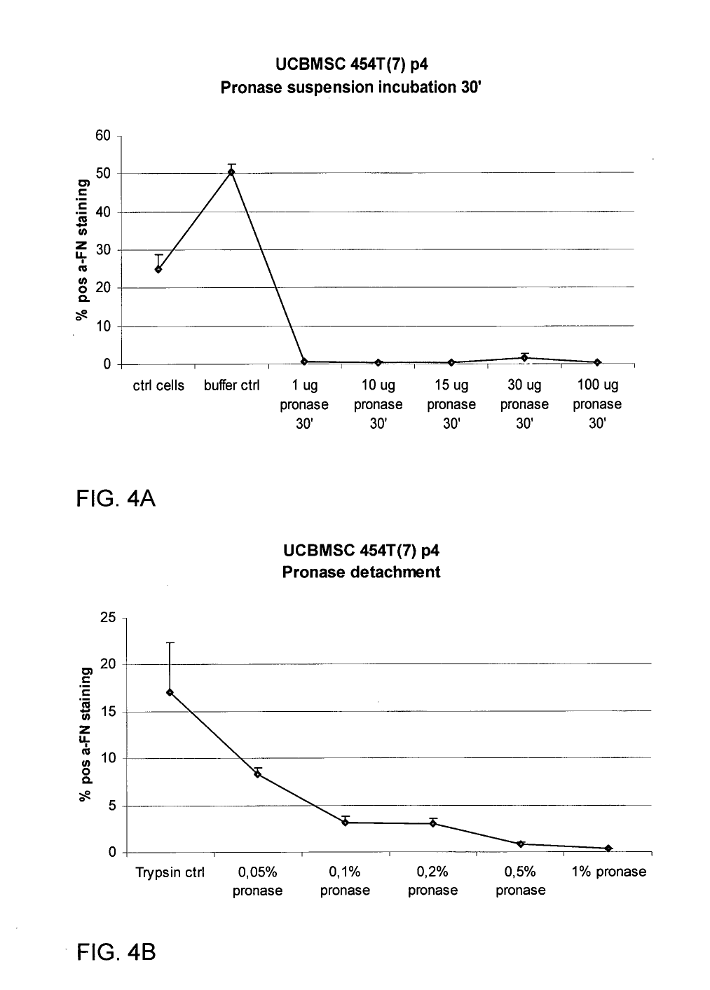

Pronase efficiently cleaves off cell surface fibronectin on MSCs after either suspension incubation of 30 min or rapid pronase detachment (<4 min) from the culture dishes (see FIGS. 4A, B). Of the tested concentrations, 30 min incubation with 1 μg pronase is effective enough to remove all cell surface fibronectin (FIG. 4A). In detachment, pronase works as efficiently as trypsin (MSC detachment in 4 min). Even the second highest concentration tested, 0.5%, detached cells with high viability, but lacking cell surface fibronectin. The highest concentration tested, 1%, affected cell morphology slightly, producing more fuzzy looking cells, although cell viability was high (>95%).

### EXAMPLE 5

### Materials and Methods

Human umbilical cord blood-derived mesenchymal stem cell (UCB MSC) line 391P in p5 and pediatric human bone marrow-derived mesenchymal stem cell (BM MSC) line M2 in passage 6 were used in the experiments. Sub-confluent cells were detached with either:
a) 0.1% pronase (Roche) in PBS w/Ca & Mg pH 7.2 with 0.5 mM EDTA
b) 0.5% pronase (Roche) in PBS w/Ca & Mg pH 7.2 with 0.5 mM EDTA
c) trypsin detachment (TryPLe Express, Invitrogen) was used as control.

Cell detachment was stopped by adding excess culture media when the cells had detached (within 4 min). Cell viability was determined with Trypan blue exclusion. The cells were pelleted by centrifugation (300×g, 5 min) and resuspended in PBS w/Ca & Mg pH 7.2+0.5% bovine serum album (BSA, ultrapure, Sigma). The cells were let to recover at room temperature for either 1.5 or 3 h. At the end of the recovery incubation, the cell viability was rechecked with Trypan blue exclusion and cells were labelled for flow cytometry with anti-fibronectin antibody (#ab6327, abcam), an extended ISCT MSC minimum criteria panel (Dominici et al. 2006) of antibodies CD13, CD44, CD49e, CD29, CD90, CD73, HLA-ABC, CD105, the negative MSC markers HLA-DR, CD34, CD45, CD14, CD19 and some antibodies against cell surface adhesion molecules: PODXL, CD49f and CD49d (FIG. 17). The cells were labelled for flow cytometric analysis with 2-3 μl of the antibodies per 1×10e5 cells in DPBS pH 7.2+0.5% bovine serum albumin (BSA) for 30 minutes on ice. After washing with excess labelling buffer, secondary antibody staining was done for the fibronectin staining with Alexa 488-conjugated goat-anti mouse IgG (H+L) diluted 1:500. The labelled cells were run with a FACSAria (BD) flow cytometer and the results were analyzed with the FACSDiva software (BD).

### Results

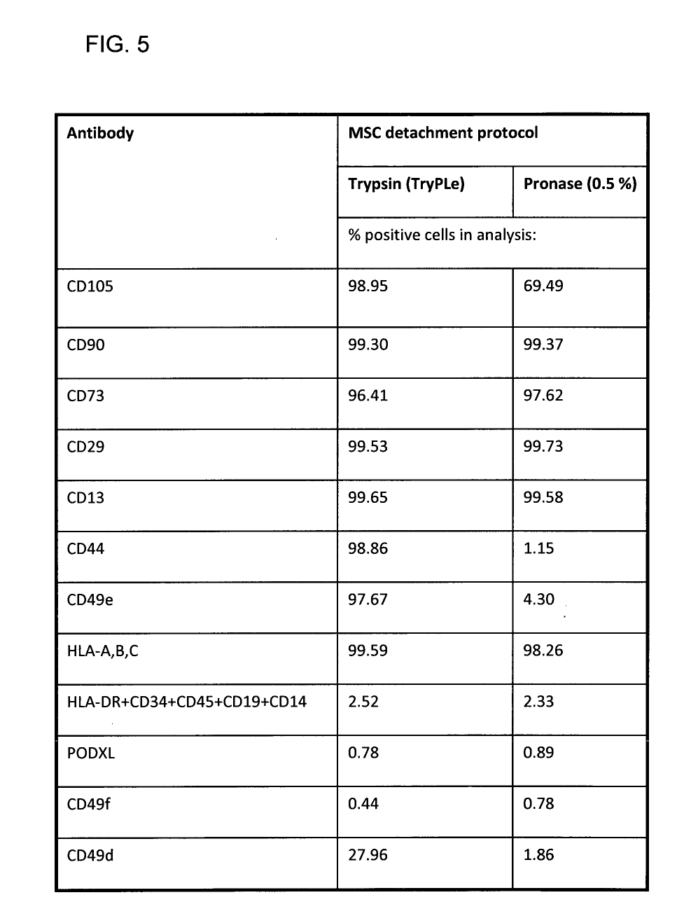

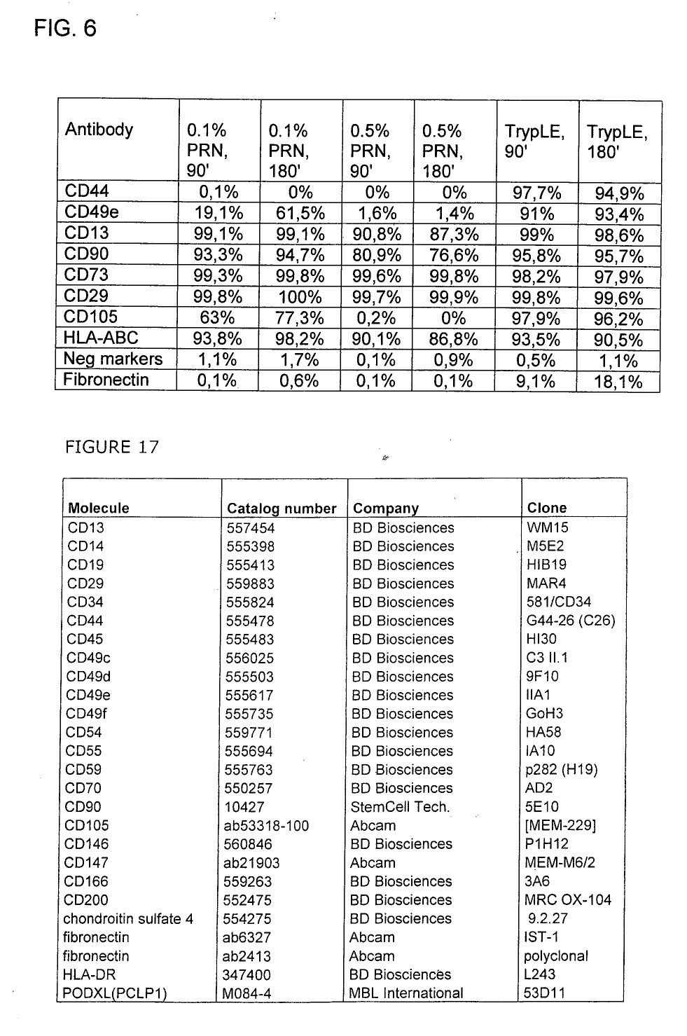

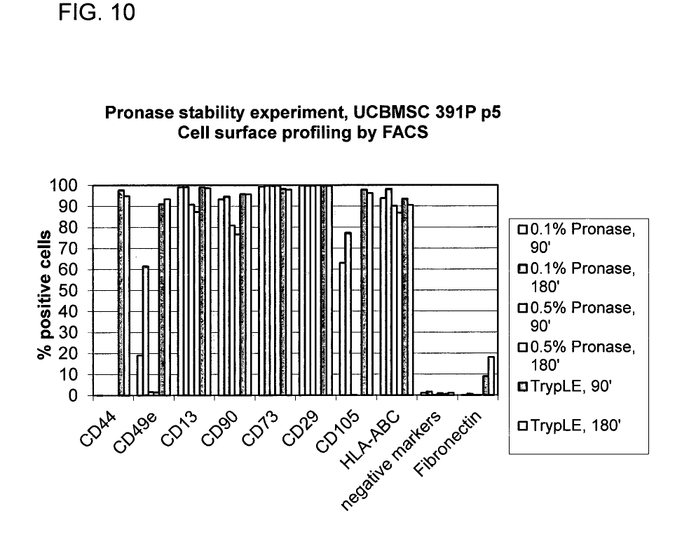

Pronase efficiently cleaves fibronectin from the cell surface, but also produce other changes in the antibody cell surface binding profile (see FIG. 5). It is noteworthy, that for instance the binding of the CD44 antibody is completely vanished, indicating a cleavage of the hyaluronan receptor CD44 (FIG. 5). Cleavage is also seen for the CD105 antigen and the integrins CD49d (α4) and CD49e (α5). The recovery tests for 90 and 180 min after either 0.1% (mild) or 0.5% (strong) pronase detachment demonstrated several interesting things concerning specificity, efficiency and stability of the pronase treatment. The pronase effect seems to be specific and dose-dependent as recovery is faster for the milder detachment for e.g. CD49e, CD13, CD90 and CD105 (see FIGS. 6 and 10). Fibronectin cleavage, however, is efficient with both concentrations and more stable than the other studied cell surface antigens, since recovery does not occur during either 90 or 180 min recovery at room temperature (see FIGS. 6 and 10). Importantly, pronase does not cleave off all cell surface proteins as demonstrated by intact expression of e.g. CD73, CD29 and the negative markers HLA-DR, CD34, CD45, CD14, CD19 (FIGS. 5, 6 and 10). These results indicate that a different cell surface can be produced by pronase detachment and for many cell surface antigens the effect is evidently transient with a recovery process initiated after 90-180 min. It is however noteworthy that the effect is more stable for fibronectin, an ECM protein (FIGS. 6 and 10). Pronase treatment could thus be used as a relevant method to produce cells without cell surface fibronectin and a transiently altered cell surface for other antigens and adhesion molecules. Additionally, the transiently altered cell surface will be compensated, but not too quickly. These results indicate that the method could thus be translated to a clinical setting. Viability was also always >95% with all tested pronase conditions and recovery times.

### EXAMPLE 6

### Materials and Methods

UCBMSC 391P in p5 was used for these experiments. The subconfluent cells were either detached with 0.1 or 0.5% pronase in PBS w/Ca & Mg pH 7.2+0.5 mM EDTA. Trypsin detachment (TryPLe Express, Invitrogen) was used as control. Detachment was stopped after 4 minutes by adding excess culture media. Viability was determined by trypan blue exclusion. Detached cells were plated at a density of 1000 cells/cm² for osteogenic and adipogenic differentiation protocols. The cells were let to differentiate for 2-3 weeks with standard differentiation protocols (osteogenic differentiation media: αMEM with 10% FCS, 50 μM dexamethasone, 1M β-glycerophosphate and 10 mM ascorbic acid-2-phosphate, adipogenic induction media: αMEM with 10% FCS, 28 mM Indomethasin, 44 μg/ml IBMX-22 (3-isobutyl-1-methylxanthine), 400 μg/ml dexamethasone (DM-200) and 0.5 mg/ml insulin −0.25, adipogenic terminal differentiation media: αMEM with 10% FCS and 28 mM Indomethasin, 0.5 mg/ml Insulin −0.25 and 3 mg/ml Ciglitazone −1.5). Control cells were cultured in standard proliferation media. Differentiated cells were stained with either von Kossa (osteogenic) or Sudan III (adipogenic) staining protocols. Representative pictures were taken with a phase contrast microscope.

### Results

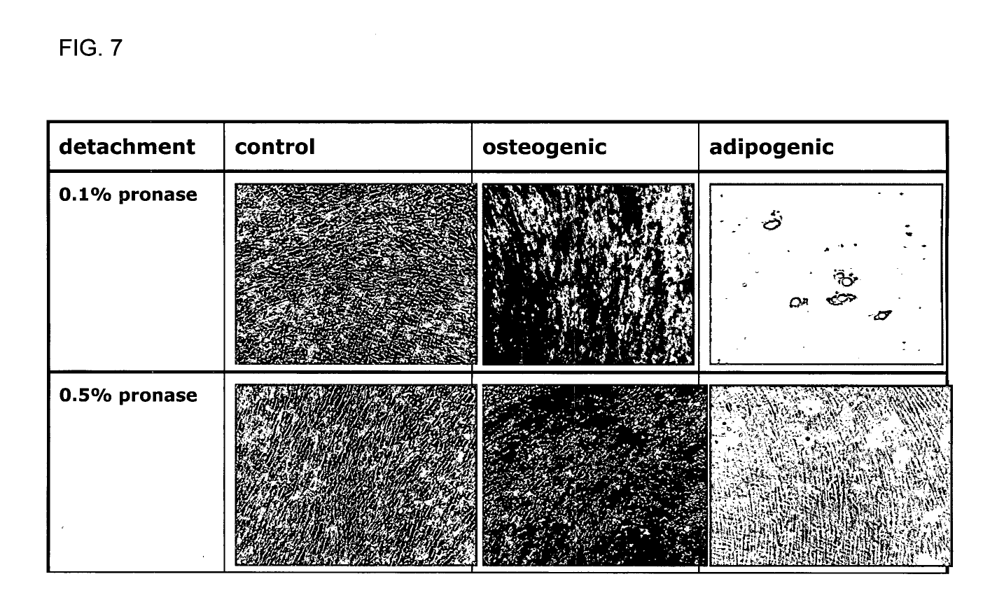

Pronase detachment (0.1 or 0.5%) did not evidently affect the multipotent potential of the MSCs, since both osteogenic and adipogenic differentiation was successful (see FIG. 7) and comparable to results received after standard trypsin detachment. Pronase does not affect MSC multipotency capacity, although transient changes in cell surface expression is evident (see FIGS. 5 and 6).

### EXAMPLE 7

### Materials and Methods

UCBMSC 588P in p4 and BMMSC 372 (28-year female donor) in p6 were used for radioactive in vivo experiments. The subconfluent cells were detached either with 0.5% pronase in PBS w/Ca & Mg pH 7.2+0.5 mM EDTA or with trypsin (Trypsin-EDTA, Sigma). Cell detachment was stopped by adding excess culture media when the cells were detached (within 4 min). Next, the stem cells were labelled with ^{99m}Tc hydroxymethylpropylene amine oxime (Tc-HMPAO, Ceretec®, Amersham Healthcare). Briefly, Tc-HMPAO was added to the stem cell suspension, and left for 15 min at room temperature. The cell suspension was centrifuged in sterile tubes at 300 G for 5 min. The supernatant was then separated from the stem cells, and the cells were resuspended in growth medium. After labelling, cell viability was determined by trypan blue exclusion.

To study the biodistribution of the radiolabelled cells, 7-8 weeks old female Athymic Foxn 1 nude mice were anesthetized using Hypnorm®-Dormicum® mixture (1 part of Hypnorm® (fentanylsitrate 0.315 mg/ml and fluanisoni 10 mg/ml), 1 part Dormicum® (midatsolam 5 mg/ml) and 2 parts of water) and either pronase or trypsin treated cells (5×10⁵ cells/mouse in 100 μl of saline) were administered via tail vein. One or 15 hours post injection mice were killed and imaged using Siemens Orbiter gamma camera (Siemens Gammasonics Inc., Des Plaines, Ill., USA)) equipped with a pin-hole collimator. After imaging, following organs and tissue samples were collected: lungs, heart, liver, spleen, pancreas, kidneys, bone, bone marrow, GI tract. Radioactivity of the samples was determined using a gamma counter (Wallac Wizard 1480, Perkin Elmer, Gaithersburg, Md., USA).

In the second set of experiments, UCB MSC cells (454T(7) in p6) were labelled with PKH-26 fluorescent dye (Sigma) and cultured for 2 days followed by detachment of the cells either with 0.5% pronase in PBS w/Ca & Mg pH 7.2+0.5mM EDTA or with trypsin (Trypsin-EDTA, Sigma). Cell detachment was stopped by adding excess culture media when the cells were detached (within 4 min). Cell viability (trypan blue exclusion) and PHK-labelling efficacy (flow cytometry) was determined prior cell injections. Cells were injected into 10 weeks old male Athymic Foxn 1 Nude mice intravenously using 1×10⁶ cells/mouse in 100 μl of saline. One hour and 20 hours after cell administration mice were killed and following organs and tissue samples were collected: lungs, spleen, peripheral blood, and bone marrow. In order to remove red blood cells, peripheral blood samples were incubated with BD FACS™ Lysing solution (BD) and remaining cells were washed FACS-buffer (PBS w/Ca & Mg pH 7.2+0.5% bovine serum album (BSA, ultrapure, Sigma)). Bone marrows were collected by washing the femurs using PBS pH7.2−2 mM EDTA+2% FCS-buffer. Lungs and spleens were homogenized with scalpels and homogenates were filtered through 70 μm filter strainers. Prior flow cytometry analysis, all the samples were diluted with FACS-buffer.

### Results

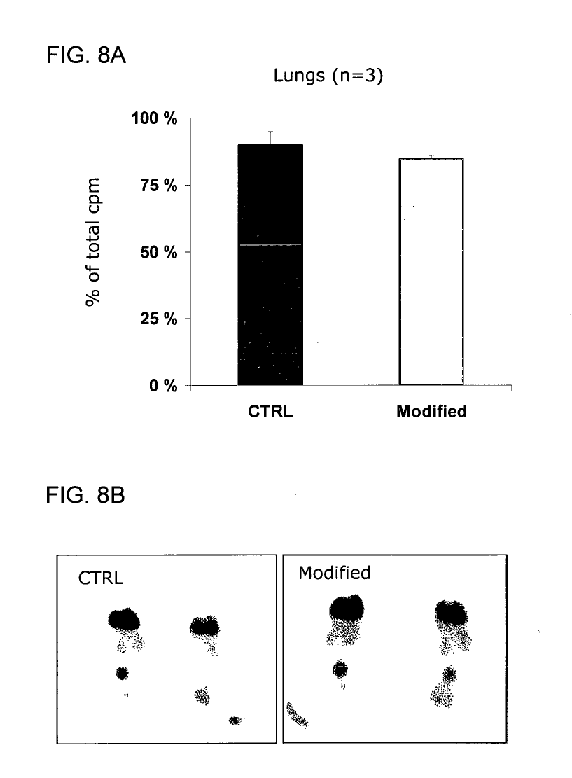

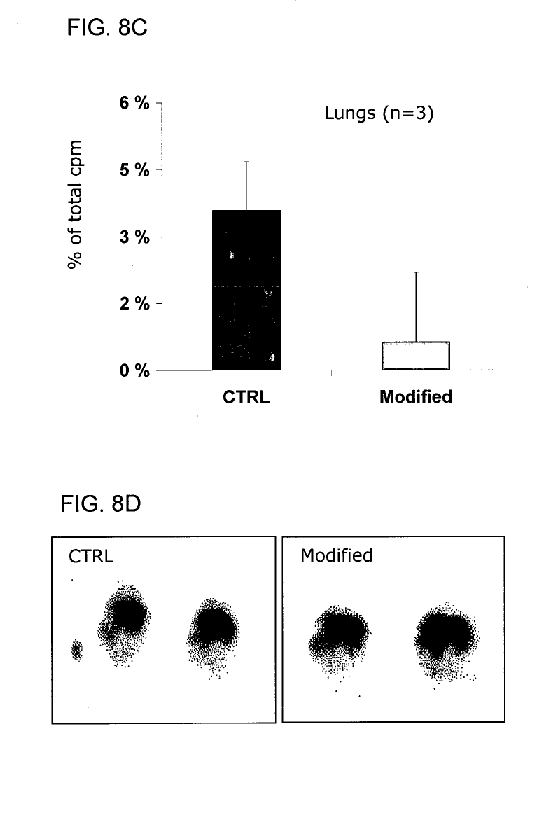

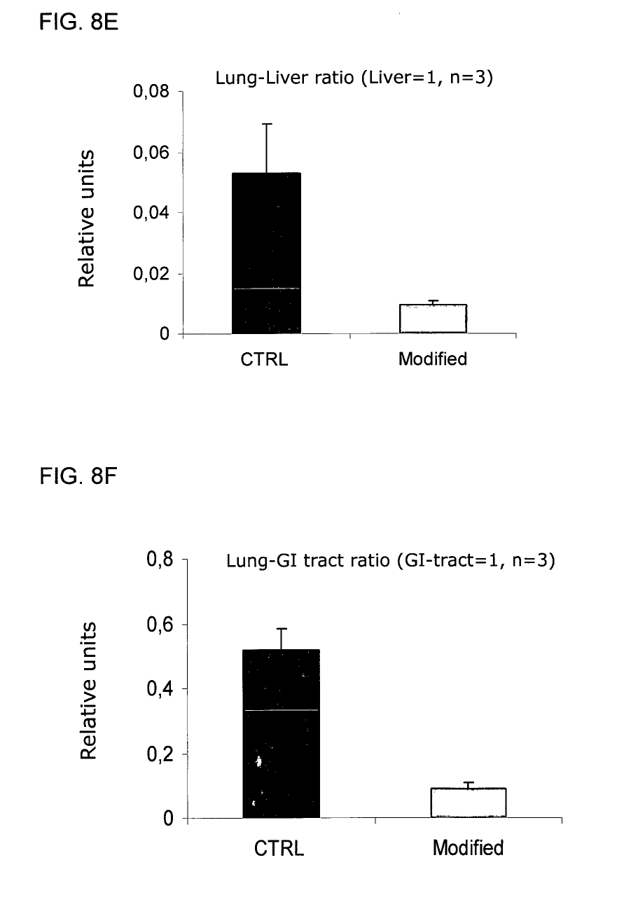

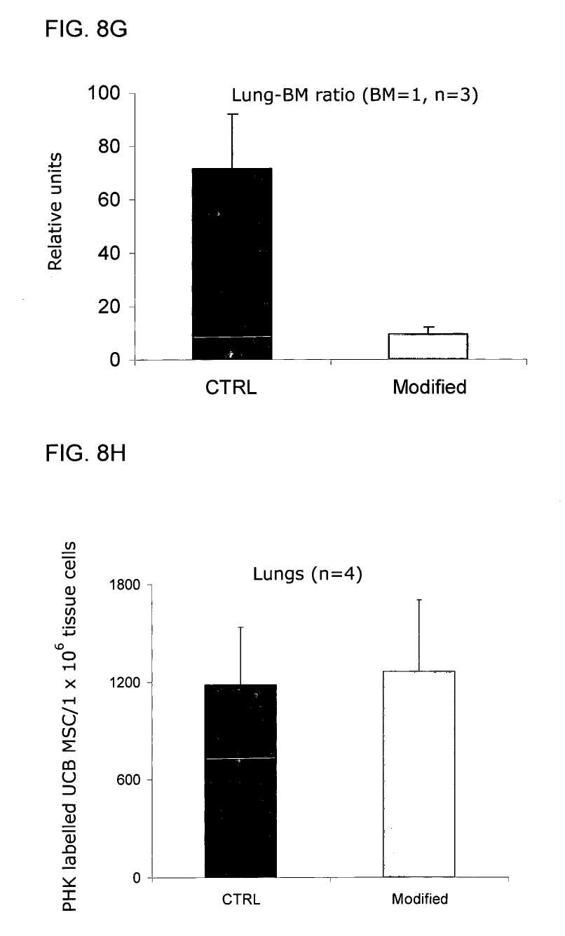

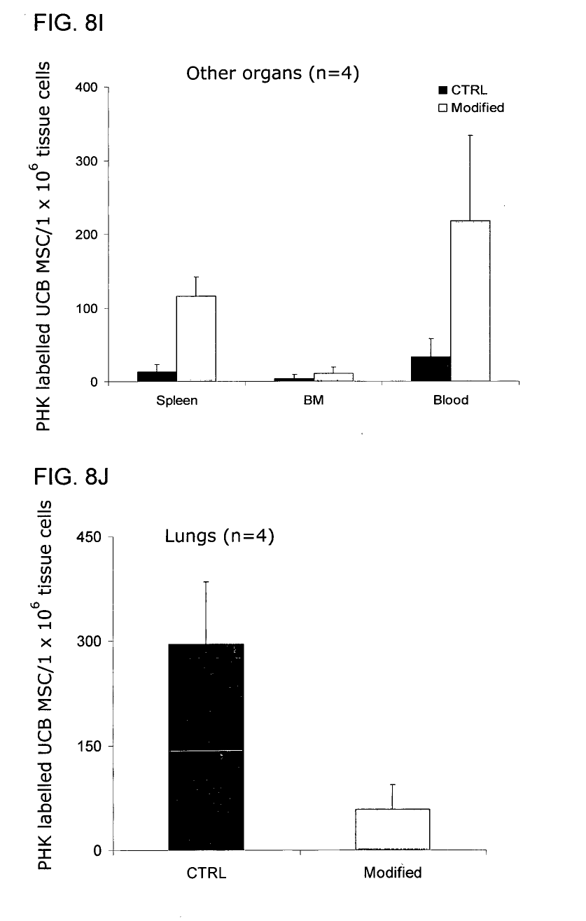

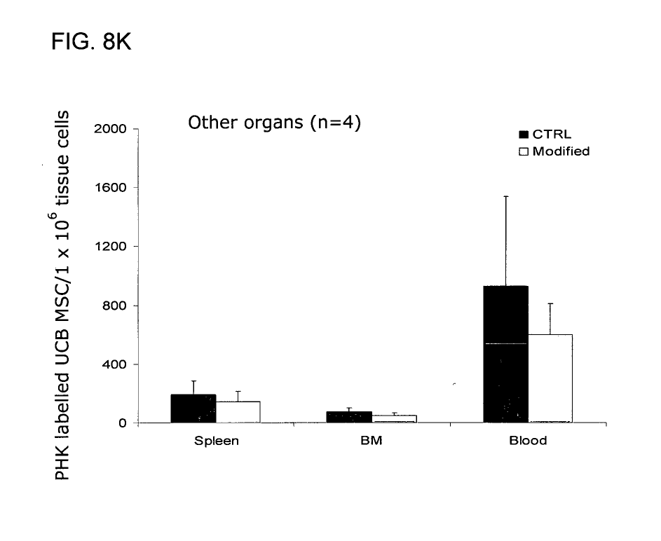

As shown in FIGS. 8A and 8B, similar amount of radioactivity (>80% of the total activity) was detected from lungs in both control and pronase treatment groups one hour after UCBMSC administration. On the contrary, when lungs were analysed 15 hours after injection (see FIG. 8C), less radioactivity was detected in pronase treatment group (0.6% of the total activity) when compared to control group (3.6%). However, the difference was not clearly seen in gamma camera imaging (see FIG. 8D), probably due to lack of sensitivity of the detection method. When compared lungs to liver, GI-tract and bone marrow (see FIG. 8E-G), again decreases amount of radioactivity was detected in pronase treatment group in comparison to control group. For example, in comparison to bone marrow, lungs were 7 times less radioactive in pronase treatment group than in control group (FIG. 8G). In the case of PKH-26 labelled UCB MSCs, 1 hour after injection there were circa 1200 PHK-26 labelled cells per million lung tissue cells in both control and pronase treatment group (see FIG. 8H). When spleen and peripheral blood were analyzed, the number of detected PHK-26 labelled cells was 9 and 6 times higher, respectively, in comparison to control group (see FIG. 8I). Twenty hours later, the number of fluorescent UCB MSCs in the lungs was 5 times lower in the pronase group when compared to control group (8J). However, there was no difference in the number of fluorescent UCB MSCs in spleen, BM, and peripheral blood between control and pronase treatment group (see FIG. 8K).

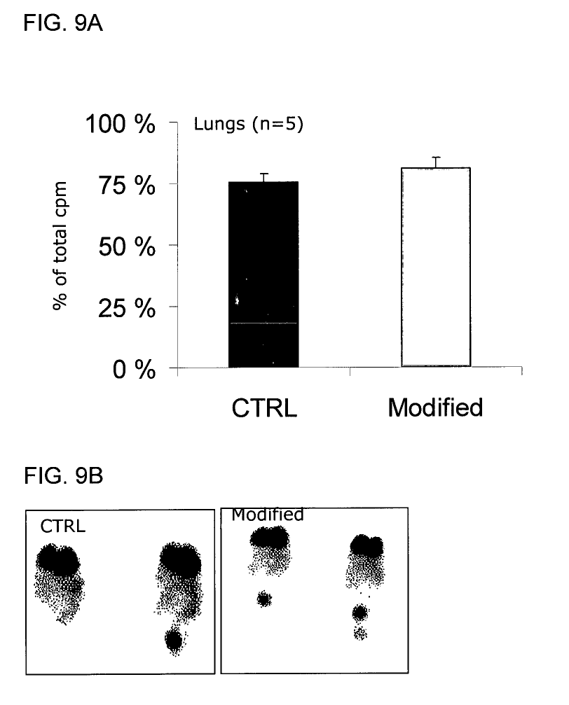

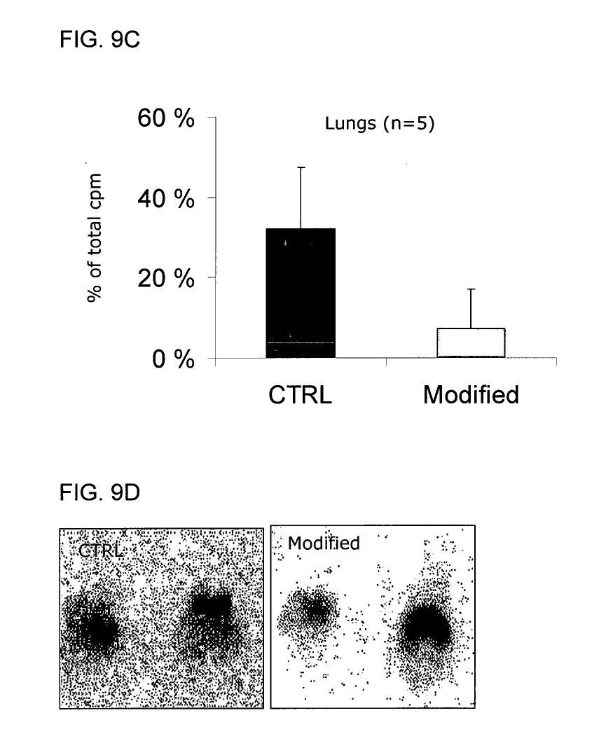

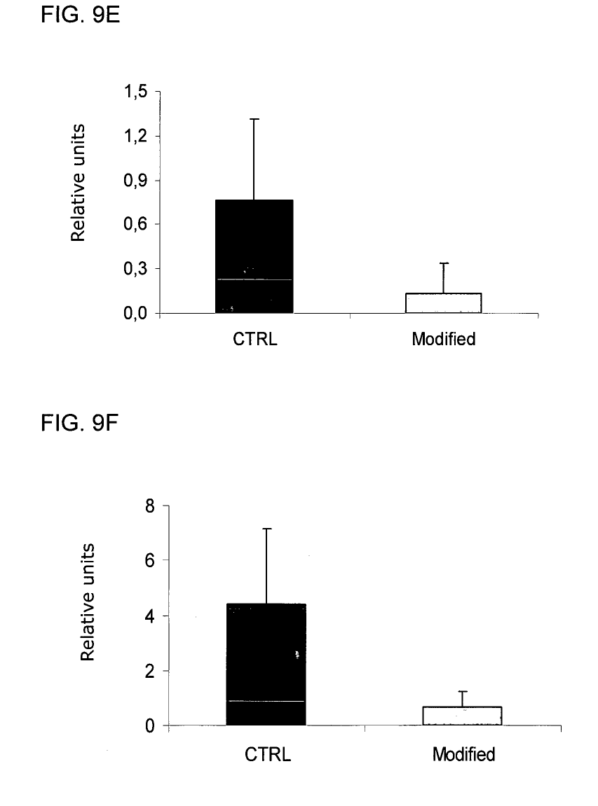

When studying the biodistribution with BMMSCs similar observations were done as with UCBMSCs. One hour post injection, 75-81% of the total radioactivity was detected from the lungs in both treatment groups (see FIGS. 9A and B). In the latter time point (15 hours post injection) the difference between pronase treatment group and control group was even more notable than in case of UCBMSCs: circa 32% and 7% of the total activity was detected from lungs in pronase group and control group, respectively (see FIG. 9C). In addition, when lungs were compared to other organs (e.g. liver and GI-tract), less radioactivity was detected from the lungs in pronase treatment group when compared to control group (FIGS. 9E-F). For example, when compared to GI-tract, circa 4 times lower radioactivity was detected from the lungs of pronase group in comparison to control group (FIG. 9F). Taken together, our results suggest that the pronase treatment can enhance the stem cell clearance from lungs.

### EXAMPLE 8

### Mass Spectrometric Analysis of Cell Surface Protein After Pronase Treatment Materials and Methods

Umbilical cord blood-derived MSC 391P at passage 4 were used for mass spectrometric analysis of cell surface proteins after pronase treatment. The subconfluent cells were treated either with 0.5% pronase (in PBS w/Ca & Mg pH 7.2+0.5mM EDTA, Roche) or as a control with trypsin (TryPLe Express, Invitrogen). Cell detachment was stopped by adding excess culture media when the cells were detached (within 4 min). Next, half of the cells (0 h time point) were processed for surface analytics samples. The second half of the cells were incubated for 5 h, at +37° C. and then processed and analysed as the 0 h time point samples. Aliquots from both time points and both detachments were also analysed by flow cytometry for the expression of CD105, CD90, CD73, CD44, CD49e, CD49d, CD55, CD59, CD200, HLA-DR, CD34, CD45, CD19, CD14, and Fibronectin (ab2413). Based on mass spectrometric analysis, selected cell surface proteins were also analysed with flow cytometry, i.e., CD166, galectin-1, chondroitin sulfate proteoglycan 4 (CSPG4), CD49c, CD146, and CD147 in UCBMSC 391P (p5). The analysis was performed similarly as described in Example 5. Galectin-1, CSPG4, CD147 and fibronectin required labelled secondary antibody.

### Cell Surface Protein Biotinylation

Biotin label (EZ-Link NHS-SS-biotin, Thermo Fisher Scientific Inc.) was resolved in D-PBS buffer (Dulbecco's phosphate buffered saline, Dulbecco). Prior to labelling the cells were washed three times with ice cold D-PBS. The cells were incubated in labelling solution on ice for 30 minutes and washed twice with ice cold D-PBS. Unreactive label was blocked by incubating with 20 mM glycine in D-PBS for 15 minutes. The cells were then washed three times with ice cold D-PBS and lysed in lysis buffer containing 2% NP-40, 1% Triton-X 100, 10% glycerol, 350 mM sodium chloride, protease inhibitors (EDTA free protease inhibitor tablet, Roche) in PBS. The cells were scraped off the plate, moved to a microcentrifuge tube and incubated on ice for 30 minutes. 2 μl of 10 U/μl DNAase (DNase I recombinant, RNase-free, Roche) was added per 50 μl of lysate and the mixture was incubated in room temperature for 50 minutes. Lysate was centrifuged 15000 rpm at +4° C. for 20 minutes. Magnetic streptavidin beads (Dynabeads MyOne Streptavidin T1, Invitrogen) were washed with lysis buffer and blocked by 1% ultra pure bovine serum albumin (BSA, Sigma-Aldrich) in lysis buffer. 0.1% of ultra pure BSA and the washed beads were added to the cell lysate and incubated in room temperature for 40 minutes. The beads were separated from the solution with magnetic stand (DynaMag-Spin, Invitrogen). The solution containing the unbound proteins was saved. The beads were washed sequentially A) three times with lysis buffer, B) twice with modified lysis buffer containing 1% NP-40, 0.5% Triton-X 100 and no glycerol, C) three times with D-PBS and D) once with water. The bound proteins were eluted from the beads by elution buffer (50 mM DTT, 25 mM Tris, pH 7.5). The eluate was saved and the elution step was repeated. The first and the second eluate were combined and vacuum dried.

In-liquid reduction, alkylation and digestion of proteins were performed as described earlier (Kinter and Sherman, 2000, Protein sequencing and identification using tandem mass spectrometry, John Wiley and Sons, New York, 161). In brief, vacuum dried proteins were resolved in 6M urea in 100 mM Tris and reduced with 200 mM DTT in 100 mM Tris. Reduced proteins were alkylated with 200 mM iodoacetamide in 100 mM Tris and unreacted iodoacetamide was consumed with reducing solution. The mixture was diluted with water prior to digestion to decrease urea concentration. 5-10% (w/w of calculate cell surface protein amount, i.e. 5% of total protein amount in the cell lysate) of trypsin (sequencing grade, Promega Ltd) was added and the reaction mixture was incubated at 37° C. over night. The reaction was stopped by adding glacial acetic acid as needed to lower pH of the solution below 6. The sample was vacuum dried and resolved in 0.1% formic acid for mass spectro-metric analysis.

To obtain comprehensive view of the cell surface proteins, samples were run in SDS-PAGE and the gel was silverstained. SDS-PAGE gel lane, which contained the proteins eluted from streptavidin beads, was cut into 2 mm slices for in-gel digestion. Each slice was cut into pieces with diameter of 0.5 mm, which were shrunk by adding twice 200 μl of acetonitrile. Gel pieces were rehydrated with 100 μl of 20 mM DTT in 0.1 M ammonium bicarbonate for 30 min at 56° C. Excess liquid was removed and the gel pieces were dehydrated as above. 100 μl of 55 mM iodoacetamide in 0.1 M ammonium bicarbonate was added and the gel pieces were incubated 15 min in dark at room temperature. Excess liquid was removed, the gel pieces were washed with 100 μl of 0.1 M ammonium bicarbonate and dehydrated as above. 0.2 μg of trypsin (sequencing grade, Promega Ltd) in digestion buffer (10% acetonitrile in 0.09 M ammonium bicarbonate) was added and plain digestion buffer if needed to cover the gel pieces completely. The gel pieces were incubated at 37° C. over night. To recover the peptides, excess liquid was collected, 25 mM ammonium bicarbonate was added to cover the gel pieces and they were incubated for 15 min at room temperature. Excess liquid was collected and the gel pieces were incubated in 5% formic acid for 15 min at room temperature. The last step was repeated and all collected supernatants were pooled.

a Mass Spectrometry

Protein digests were analysed with liquid chromatography (LC)—mass spectrometry (MS). Peptides were loaded to reversed phase precolumn (NanoEase Atlantis dC18, 180 μm×23.5 mm, Waters) with 0.1% formic acid and separated in reversed phase analytical column (PepMap 100, 75 μm×150 mm, Dionex Corporation) with linear gradient (4-50%) of 95% acetonitrile in 0.08% formic acid in 40 minutes. Ultimate 3000 LC instrument (Dionex Corporation) was operated in nano scale with flow rate of 0.3 μl/min. Both precolumn and analytical column were placed in column oven at 30° C. Eluted peptides were introduced to LTQ Orbitrap XL mass spectrometer (Thermo Fisher Scientific Inc.) via ESI Chip interface (Advion BioSciences Inc.) in positive-ion mode.

The mass spectrometer was calibrated with Thermo Fisher Scientific standard LTQ calibration solution consisting of caffeine, MRFA tetrapeptide and Ultramark 1621. The instrument was tuned with glu-fibrinopeptide B (Sigma-Aldrich). Full scan for eluting peptides was acquired in mass range of 300-2000 m/z on Orbitrap-detector with 60 000 resolution at 400 m/z, AGC target set to 200 000 and maximum inject time set to 800 ms. Based on full MS scan, six MS/MS data-dependent scans were acquired on LTQ with AGC target set to 10 000 and maximum inject time set to 100 ms. Isolation width of 2 m/z was used for precursor selection. Normalized collision energy of 35%, activation time of 100 ms and activation Q set to 0.25 were used in peptide fragmentation. Precursors, whose charge state couldn't be determined or charge state was +1, were discarded from MS/MS analysis. Precursors were dynamically excluded for 10 s with repeat count of 1. Both full MS scan and MS/MS scans consisted of one microscan and they were acquired as a profile data.

### Mass Spectrometric Data Analysis

Data files from mass spectrometer were processed with Mascot Distiller (Matrix Science Ltd., version 2.2.1.0) via Mascot Daemon Client (Matrix Science Ltd., version 2.2.2). Full scan was considered valid if it contained at least one peak. Precursor charge state and m/z value were re-determined from parent scan. Maximum precursor charge of +7 was allowed to have corresponding MS/MS scan being included in analysis. Following parameters were used for peak picking from full scan: minimum signal-to-noise ratio 5; minimum peak width 0.001 Da; expected peak width 0.05 Da and maximum peak width 1 Da.

Each MS/MS scan needed to have at least ten peaks and maximum charge of +2 was allowed for fragment ions. MS/MS scans were aggregated if precursor m/z values matched with +/−0.02 m/z tolerance and elution time difference was less than 30 s. Following parameters were used for peak picking from MS/MS-scan: minimum signal-to-noise ratio 1; minimum peak width 0.01 Da; expected peak width 0.4 Da and maximum peak width 1 Da.

Processed data was searched with Mascot Server (Matrix Science Ltd., version 2.2.04) against human proteins in either UniRef100 database (version 14.6) or UniProt database (version 14.6). Search parameters were as follows: enzyme trypsin (normal cell surface proteome analysis) or semitrypsin (in experiments comparing pronase and trypsin release); maximum missed cleavages 1; variable modifications: lysine 3-(carbamidomethylthio)propanoylation, protein N-terminal 3-(carbamidomethylthio)propanoylation, cysteine carbamidomethylation, methionine oxidation, cysteine propionamidation (in-gel digests only); peptide mass tolerance +/−0.02 Da; fragment mass tolerance +/−0.8 Da and instrument type ESI-TRAP.

LC-MS differential expression analysis was performed with Progenesis LC-MS software (Version 2.0, Nonlinear Dynamics Ltd.), where also technical replicates were compared.

In the pronase treatment experiment, the data was exported from the Progenesis LC-MS files as an .csv feature table, which was further processed manually using Microsoft excel software. The following processing steps were performed in the following order: 1) Peptides occuring either with higher intensity at Pronase or Trypsin samples were separated into different cathegory, 2) Peptides with anova <0.05 were selected, 3) Peptides with Mascot score <20 were neglected, 4) Peptides from Albumin, Keratin and FCS derived proteins or from proteins with distinct inner cellular location (Actin, Tubulin, Heat shock proteins, Histones) were neglected. Further protein results were classified according to the peptide number found in different sample sets and visualized.

### Results

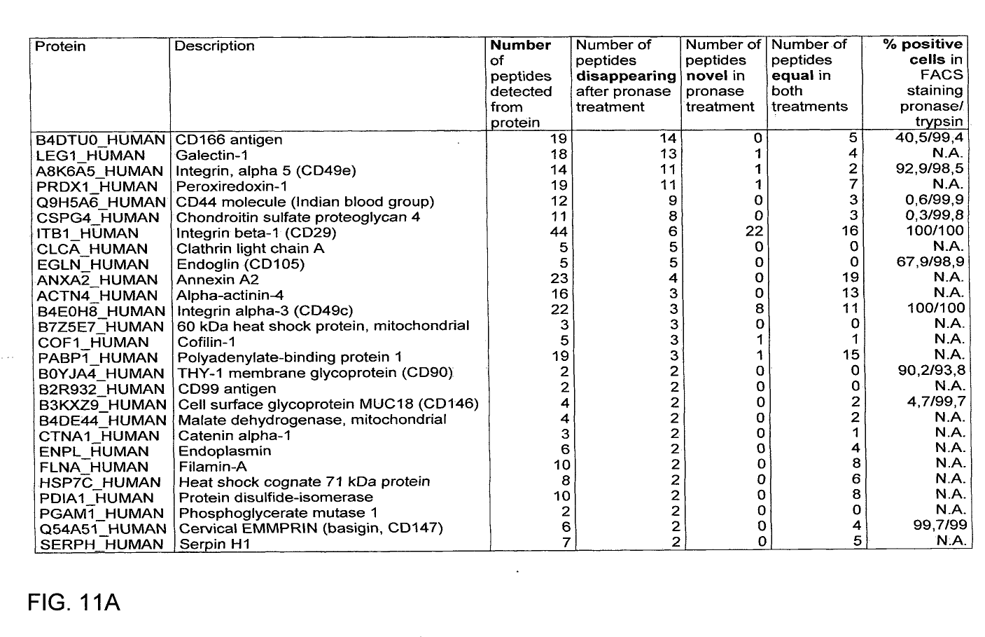

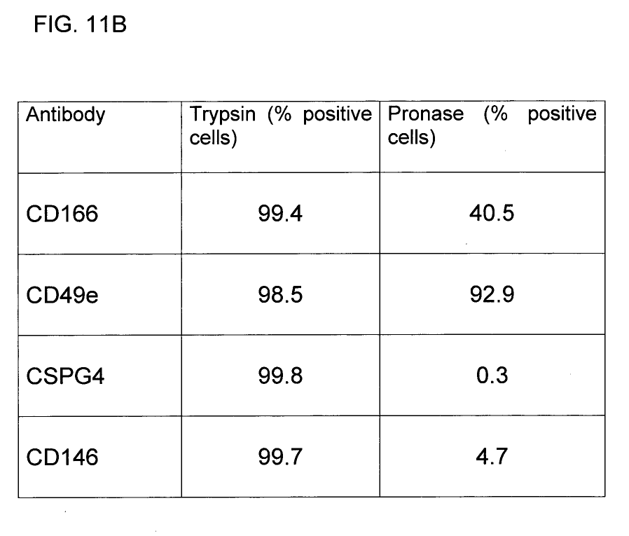

A list of cell surface proteins of MSC that were digested by the pronase treatment are shown in FIG. 11A. Proteins CD166, galectin-1, integrin alpha 5, CD44, chondroitin sulfate proteoglycan 4 (CSPG4), endoglin (CD105), THY-1(CD90), CD99, and MUC18 (CD146) all had a higher number of peptide fragments disappearing after the pronase treatment than after the control trypsin treatment. The peptide fragments here refer to the initially identified peptides. Is is of note that also some other proteins were cut by pronase but their known functions were not related to cell homing or adhesion.

To confirm the LC-MS data, we performed a flow cytometric analysis of disappearance of antibody epitopes for selected cell surface proteins. Indeed, the antibody epitopes for CD166, CSPG4, and cell surface glycoprotein MUC18 (CD146) were seen to be digested by pronase (FIG. 11A, column on the right, FIG. 11B). Furthermore, CD44 was constantly digested by pronase, as were also integrin alpha 5 (CD49e) and endoglin (CD105).

### EXAMPLE 9

### Materials and Methods

Four enzymes were tested whether they produce cell surface changes similar to pronase. Tested enzymes were 1) elastase (Merck 324682-1000 U Lot D00089585, 55 U/mg, 10 mg/ml in PBS), 2) subtilisin (Calbiochem 572909-100 mg Lot D00088518, 490 U/mg, 10 mg/ml in PBS), 3) proteinase K (Calbiochem 539480-100 mg Lot D00089137, 49.2 U/mg), and 4) kallikrein (Sigma-Aldrich, K3627-1KU, 10 mg/ml in PBS). Human BMMSC and UCBMSC were used for testing.

First, all the enzymes were tested with BMMSC (lines 414, p4, 415, p3 or p4, 418, p3, 419, p3, 424, p3, and 437, p3) whether they were suitable for cell detachment. The cells were subcultured on standard cell culture surface in a 24-well format and allowed to attach/proliferate for 1-20 days. The testing was performed at least by five different concentrations for each enzyme. After the detachment, the cells were counted with Trypan blue to estimate the amount of dead cells. After counting the cells, the cells were seeded and their attachment to the cell culture surface was confirmed by eye the next day. For flow cytometry analysis the BMMSC (line 428, p5 and 437, p3-5) were cultured in cell culture flasks 1-3 weeks. The cells were either detached with test-enzyme or detached with trypsin and treated with test-enzyme if the enzyme was not suitable for detachment (in case of kallikrein). The cells were stained with anti-CD105, anti-CD90, anti-CD73, anti-CD44, anti-CD49e, anti-CD49d, anti-Fibronectin, anti-CD54, anti-CD55, anti-CD59, anti-CD200, and negative controls (for UCBMSC) anti-HLA-DR, anti-CD34, anti-CD45, anti-CD19, and anti-CD14 (FIG. 17). Adequate isotype control antibodies were also used. The labelled cells were run with a FACSAria (BD) flow cytometer and the results were analyzed with the FACSDiva software (BD).

UCBMSC 391P (p6) were cultured to 70% confluency and detached with elastase, proteinase K, subtilisin A, and trypsin and finally analysed with flow cytometry for the expression of above mentioned markers. Kallikrein was not suitable for cell detachment and was thus excluded from the experiments with UCBMSC.

### Results

BMMSC:

Proteinase K and Subtilisin were able to detach the cells if concentrations above 10 μg/ml were used. Elastase required 400 μg/ml or more for cell detachment and still a proportion of the cells remained attached. Cell viabilities were comparable to trypsin treated cells. Kalligrein did not detach the cells.

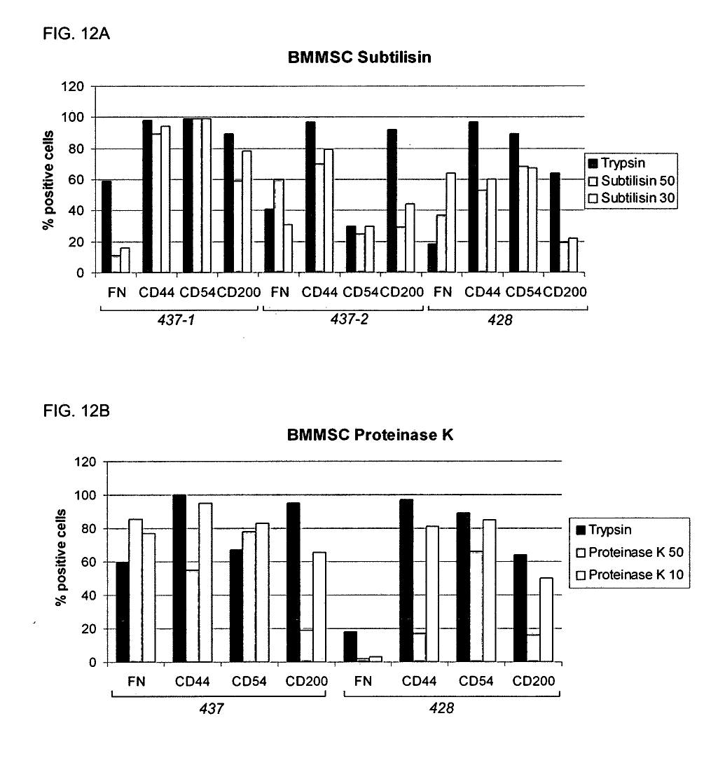

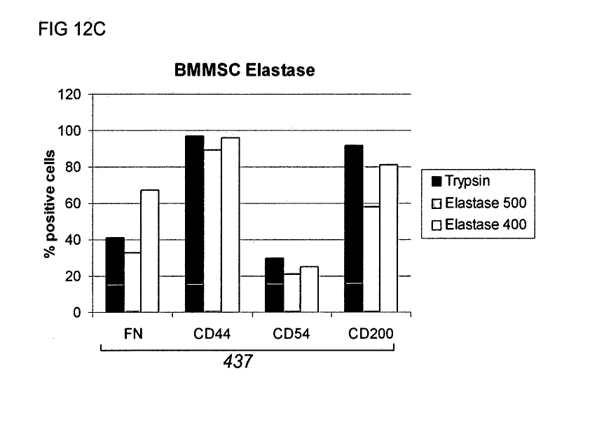

The cells surface analysis with flow cytometry revealed that none of the enzymes could modify the cell surface in the exactly same way as pronase. The expression of CD105, CD90, CD73, CD49e, CD49d, CD55, and CD59 remained similar to trypsin with all the test enzymes. The results are shown only for those proteins that had some changes compared to control (FIG. 12). Subtilisin (10 and 50 μg/ml) maintained the cell surface almost similar to trypsin. Minor decrease could be seen in CD44 and CD200 amounts (FIG. 12A). In one tested cell line (437), significant decrease in fibronectin could be seen, but when the line was tested again, the difference could not be seen. For proteinase K (10 and 50 μg/ml), most of the analysed cell surface markers had similar expression than in trypsinized cells but CD44 and CD200 were reduced compared to trypsin treated samples (FIG. 12B). Also fibronectin was removed from the cell surface in the other line (428) studied while in the other it remained similar to trypsin (437). A dose response could be seen for CD44 and CD200. The cell surface in elastase treated (400 and 500 μg/ml) cells remained similar to trypsinized cells (FIG. 12C). Kallikrein slightly reduced the amount of fibronectin on cells surface compared to trypsin (−20%) (data not shown), but as mentioned, was not suitable for detaching the cells.

UCBMSC:

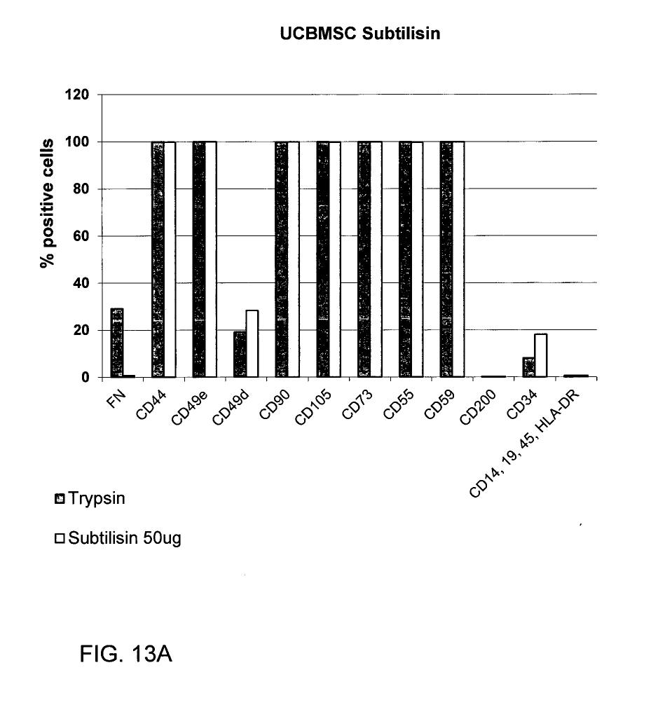

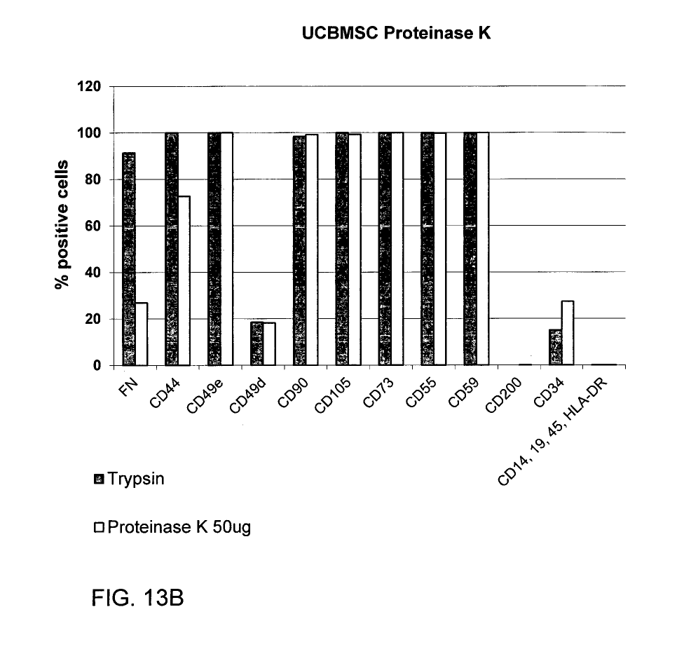

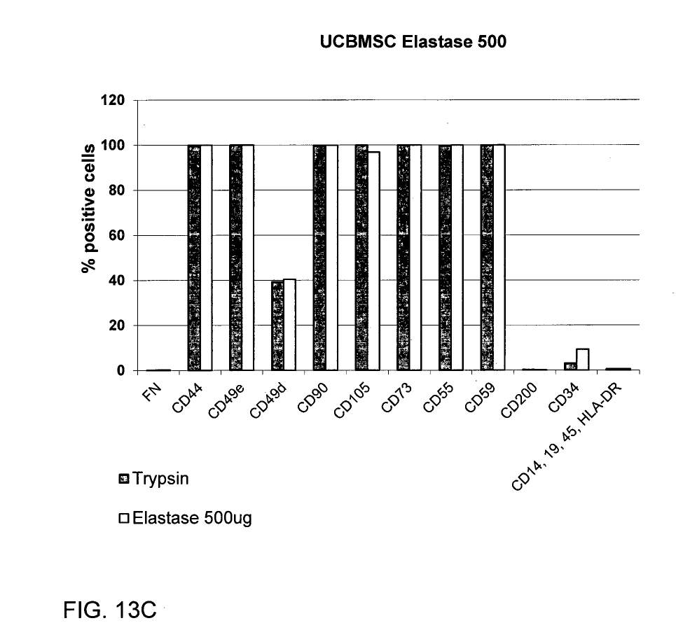

In 391P, none of the four tested enzymes produced cell surface fingerprint, which would be identical with the pronase fingerprint. The expression of most of the proteins remained similar to trypsin (FIG. 13). However, two of the tested enzymes, subtilisin and proteinase K, did cause partly similar changes on the cell surface when compared to pronase. Subtilisin decreased the amount of fibronectin (from 29.1% positive to 0.7%) (FIG. 13A) and proteinase K decreased the amount of fibronectin (from 91.4% to 26.9%) and CD44 (from 100% to 72.7%) (FIG. 13B). When testing elastase, the amount of fibronectin was low with both trypsin control and elastase indicating that the amount of fibronectin on cell surface varies maybe due to culture conditions or may detach from the cell surface during culture or washing steps (as already discussed in Example 1). Kallikrein did not detach the cells properly and was not tested with UCBMSC.

### EXAMPLE 10

### Materials and Methods

The mononuclear cells (MNC) were isolated from buffy coats from healthy anonymous blood donors (BCL23 and BCL24, Finnish Red Cross Blood Service). 40 ml of buffy coat was diluted with 100 ml phosphate buffered saline (PBS) pH 7.2 and MNCs was isolated by density gradient centrifugation on Ficoll-Pague plus (GE Helthcare, Piscataway, N.J., USA). Cells were washed with phosphate buffered saline, pH 7.2. The cell number and viability were measured from 1:20 dilution by NucleoCounter (Chemometec). 20×10⁶ cells were used to assay the T-cell proliferation of fresh cells.

In T-cell proliferation assay freshly isolated MNCs were labeled with CFSE (5(6)-Carboxyfluorescein diacetate N-succinimidyl ester). To achieve single cell suspension the cells were filtered with 30 μm sterile syringe filter (Becton Dickinson, Franklin lakes, N.J., USA). The filtered cells were resuspended in 0.1% human serum albumin (HSA, Sanquin)-PBS at the concentration of 20×10⁶ cells/ml. The same volume of freshly diluted 5 μM CFSE-solution (Molecular probes) in 0.1% HSA-PBS was added. The cells were vortexed immediately and incubated for 5 minutes at room temperature. The labeled cells were washed in 10× volume of 0.1% HSA-PBS and resuspended in RPMI growth medium at the concentration of 5×10⁶. 1.5×10⁶ CFSE-labeled cells were plated in 300 μl on cell culture treated 48 multidish plate (Nunc, Thermo Fisher). To activate T-cell proliferation 100 ng/ml CD3 antibody clone Hit3a (BioLegend, San Diego, Calif., USA) diluted in RPMI growth medium was added. Non-stimulated CFSE-labeled cells as well as stimulated and non-stimulated non-labeled MNCs were used as controls. RPMI growth medium was added to wells to achieve final volume of 650 μl/well. Plates were placed in incubator (+37° C. 5% CO₂ humidified incubator) for four days after which the cell proliferation was analyzed by using flow cytometry (FACSAria, Becton Dickinson) and FlowJo software (version 7.6.1).

MSC cell culture and co-culture assay: Trypsinized or pronase detached (0.5% pronase in PBS w/Ca & Mg pH 7.2+0.5 mM EDTA) UCBMSC (391P in p4 and UCBMSC 588P(1) in p5) were centrifuged 300×g 5 min and resuspended in RPMI growth medium at concentration of 0.5×10⁶ cells/ml. MSCs were further diluted in RPMI growth medium to achieve concentrations 2.5×10⁵ and 1×10⁵. 300 μl of each concentration was plated on 48 multidish plates (Nunc) in triplicates. The MSCs were allowed to attach two hours before adding CFSE labeled mononuclear (responder) cells. MSCs were co-cultured with MNCs for four days after which the MNCs were collected and analyzed with flow cytometry.

In addition, aliquot of UCBMSC used in the experiment were stained with anti-CD105, anti-CD90, anti-CD73, anti-CD44, anti-CD49e, anti-CD49d, anti-Fibronectin, anti-CD59, anti-CD55, anti-CD200, anti-HLA-DR, anti-CD34, anti-CD45, anti-CD19, and anti-CD14 antibodies and were analyzed with flow cytometry as described in Example 5 in order to ensure the surface modification caused by pronase treatment.

### Results

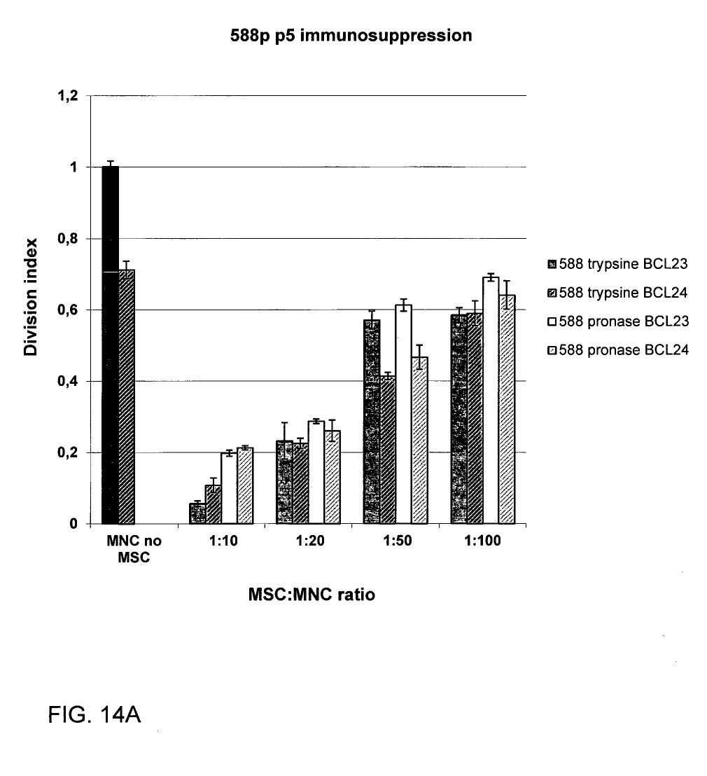

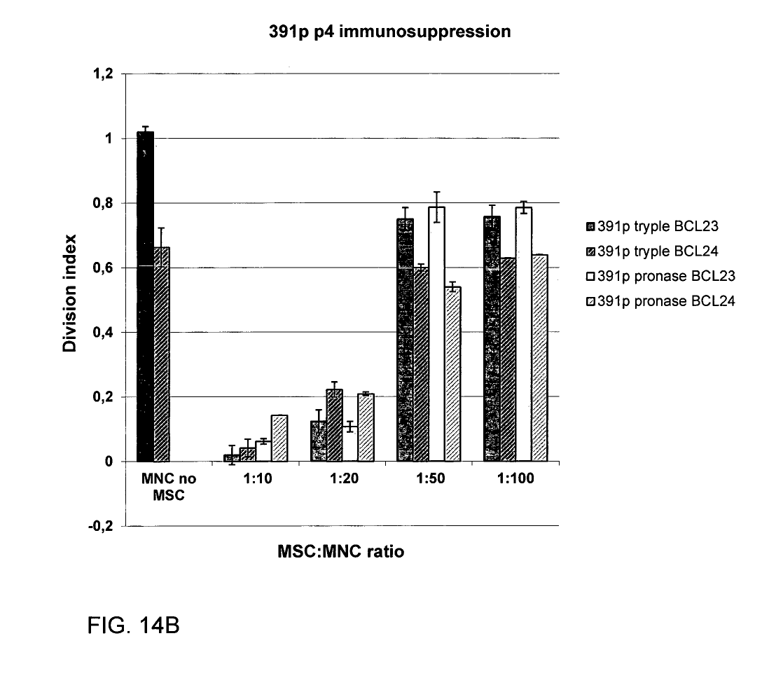

Based on the result of immunosuppression assay, the pronase treated cells inhibited the proliferation of activated T-cells as effectively as the control (trypsin treated) cells. That is, they were immunosuppressive, suggesting that the pronase treatment had no negative effect on the functionality of the cells (FIG. 14). This effect was seen with MNCs from two individuals (BCL23 and BCL24) and with two different UCBMSC lines.

When the same cells were analyzed with flow cytometry for the cell surface protein expression, typical “pronase profile” was seen. As a result of the pronase treatment, the amount of CD44, fibronectin and CD49d was decreased in comparison to trypsin treated cells. Furthermore, the intensity of CD49e was decreased (data not shown).

### EXAMPLE 11

### Materials and Methods

Cells: UCB MSC (391P, p5) and BM MSC (081, p5) cells were studied for angiogenic capacity in a validated angiogenesis model. In co-culture model, the BJ human fibroblasts purchased from ATCC (American Type Culture Collection, LGC Promochem AB, Boras, Sweden, ATCC Cat. No. CRL2522, www.atcc.org) and endothelial cells (HUVEC) isolated from human umbilical cord veins (enzymatically with 0.05% collagenase) were used.

Mediums: Mediums used in the assay were; basic test medium (BTM) (EBM-2 Basal Medium, Cat. No. CC-3156, Lonza Group Ltd., Basel, Switzerland, supplemented with 2.0% FBS, 0.1% GA and 1% L-glutamine), stimulation medium for positive test controls (BTM supplemented with VEGF and FGF2), bone marrow stem cell culture medium (BMSCM) and umbilical cord blood stem cell culture medium (UCBSCM) both prepared according to SOPs by FRCBS.

Controls: Positive controls were used to ensure that the test is technically valid. Positive control had four parallels in each test layout. Positive control had to give minimum value of 6 from 3 out of 4 parallels. Negative control was used for angiogenesis co-culture assay to ensure the technical validity of the angiogenesis assay. Negative control was the same as basic test medium (BTM). There were also cell specific media as negative controls, either BMSCM or UCBSCM, with two parallel wells in each test layout. Angiogenesis assay: BJ fibroblasts and HUVECs were co-cultured on 48 well plates for 7 days. Then on day 7, MSCs (UCB or BM), detached with either 0.5% pronase (in PBS w/Ca & Mg pH 7.2+0.5 mM EDTA) or trypsin (TryPLe Express, Invitrogen) at 70% confluency, were plated on top of the co-culture system. There were also two separate time points, i.e., MSCs were plated either right after modification (0 h) or after an incubation period (5 h in 37° C.) onto angiogenesis assay. BMMSC were plated at the density of 35 000 cells/cm² and UCBMSC at the density of 40 000 cells/cm². In addition, UCBMSC (0 h timepoint), control and pronase-treated, were also plated in inserts and angiogenic effect was studied in the same way as in co-cultures. On day 13, i.e. 6 days after adding MSCs, tubules were visualized with immunocytochemical staining using anti von Willebrand Factor (anti-vWF produced in rabbit). Cells were fixed with 70% ethanol, permeabilized with 0.5% Triton X100 and blocked for unspecific staining with 10% BSA. After blocking, the cells were incubated with primary antibody (1:5000, diluted in 1% BSA in PBS, 120 μl per well) at 4° C. overnight. Cells were then incubated 30 min at room temperature with secondary antibody (Biotinylated AntiRabbit IgG, 1:500). The result was visualized with ABCkit (avidinbiotin-complex) and DAB substrate kit. Alternatively, for immunofluorescence staining, cells were incubated with primary antibody (1:200, diluted in 1% BSA in PBS, 120 μl per well) at 4° C. overnight. Cells were then incubated 30 min at room temperature with secondary antibody (AntiRabbit IgG TRITC, 1:100 or 1:50).

Reading of the results: After immunocytochemical staining, the results were read with Nikon Eclipse TS100 microscope (Nikon, Tokyo, Japan) or Nikon Ts-i microscope (Nikon, Tokyo, Japan). Each well was visually analyzed for tubular structures and values from 0 to 8 were given for different degrees of tubule formation (two extremes being: 0=Negative control, no tubule development, endothelial cells as epithelial-like round areas in co-culture, whereas 8=Cells form dense and long tubule-like structures connecting to each other and have extensive branching that cover the whole area of well). The results were filled in the analysis chart and used for statistical analysis. The statistical analysis was performed with with GraphPad Prizm 5.0. by using one-way analysis of variance with Bonferroni's post test. The final results are reported as mild inducer (approximately 30% or less), moderate inducer (40-60%) or strong inducer (approximately 70% or more), when compared to the respective control treatments.

Aliquots of UCBMSC (0 h and 5 h) were stained with anti-CD105, anti-CD90, anti-CD73, anti-CD44, anti-CD49e, anti-CD49d, anti-CD55, anti-CD59, anti-CD200, anti-HLA-DR, anti-CD34, anti-CD45, anti-CD19, anti-CD14, and anti-Fibronectin antibodies and were analyzed with flow cytometry as described in example 5 in order to ensure the surface modification caused by pronase treatment.

### Results

The standard BM MSC and UCB MSC were able to stimulate angiogenesis in in vitro co-culture model. The effect was better in co-culture set-ups, compared to inserts, i.e., the tubule formation was the most effective when the test cells were grown in contact with the angiogenesis assay cells. The culture medium used had a strong influence on the results and was always used taken into account when calculating the results. In general, it can be concluded that MSCs were angiogenic, both after pronase-detachment and trypsin-detachment.

BMMSC: As a summary, BMMSC control cells caused a strong induction 70% more angiogenesis than in control) in BTM at 0 h, a moderate induction (40-60% more angiogenesis than in control) in BTM at 5 h and a mild induction 30% angiogenesis than in control) in their own BM medium at 5 h. Pronase-treated BMMSC caused moderate induction in BTM at 5 h and mild induction in BTM at 0 h and BMSCM at 5 h (FIG. 15A).

UCBMSC: The pronase-treated UCBMSC cultivated in the UCBSC medium at 0 h time point induced extensive angiogenesis. UCBMSC required a medium rich in growth factors in order to stimulate the tubule formation. This medium alone was also favourable to angiogenesis, as it contained inductive growth factors, e.g. PDGFBB, that has been shown to be a mild inducer in this angiogenesis assay. Besides this strong induction in UCB medium at 0 h, pronase-treated UCBMSC caused a moderate induction in UCB medium at 5 h and mild induction in BTM at 0 h. UCBMSC control cells caused a moderate induction in UCB medium at 0 h and 5 h, and in BTM at 5 h.

### EXAMPLE 12

### Materials and Methods

Cultured allogeneic porcine BMMSCs were used for the in vivo experiments. They were isolated from bilateral tibiapunction sample. The cultured cells were treated with either trypsin (control) or pronase. Prior to injection cells were labelled with 99 mTc hydroxymethylpropylene amine oxime (Tc-HMPAO, Ceretec®, Amersham Healthcare). Briefly, Tc-HMPAO was added to the stem cell suspension, and left for 15 min at room temperature. The cell suspension was centrifuged in sterile tubes at 300 G for 5 min. The supernatant was then separated from the stem cells, and the cells were resuspended in growth medium. Cells were injected i.v. in the right atrium, using 0.5 million cells/kg. Two pigs received trypsin detached cells and two pigs pronase detached cells.

Radioactivity was determined eight hours post-injection by imaging anesthetized pigs using Siemens Orbiter gamma camera (Siemens Gamma-sonics Inc., Des Plaines, Ill., USA) equipped with a pin-hole collimator. In addition, whole body SPECT-CT images were captured. After imaging, pigs were sacrified and biopsies from the following organs and tissues were collected: lungs, cerebrum, cerebellum, thymus, heart, liver, spleen, kidneys, and mesentery. The biopsies were always taken from the same anatomic location, representative of the tissue. The radioactivity of the samples was determined using a gamma counter (Wallac Wizard 1480, Perkin Elmer, Gaithersburg, Md., USA). Radioactivity was also always determined from the aliquots of labeled cells, 50000 and 100000 cells, to check the labeling of the cells. For each organ, original radioactivity per gram tissue was calculated based on the known half-life of Technetium. Radioactivity values for each organ were compared to radioactivity in the liver and represented as organ-to-liver ratio. In addition, ^{99m}Tc-BMMSC syringe content before and after injection was always measured to control the input dose.

In addition, porcine cells were analysed using flow cytometry to test the surface expression of CD44, fibronectin, CD29, CD105, CD46 and CD31 after pronase treatment. The analysis was done essentially as described in Example 5.

### Results

When BMMSCs were injected i.v. the same biodistribution phenomenon was seen as in mice. The vast majority of the radioactivity was detected in the lungs 8 hours after injection, indicating the accumulation of the injected MSC into the lungs.

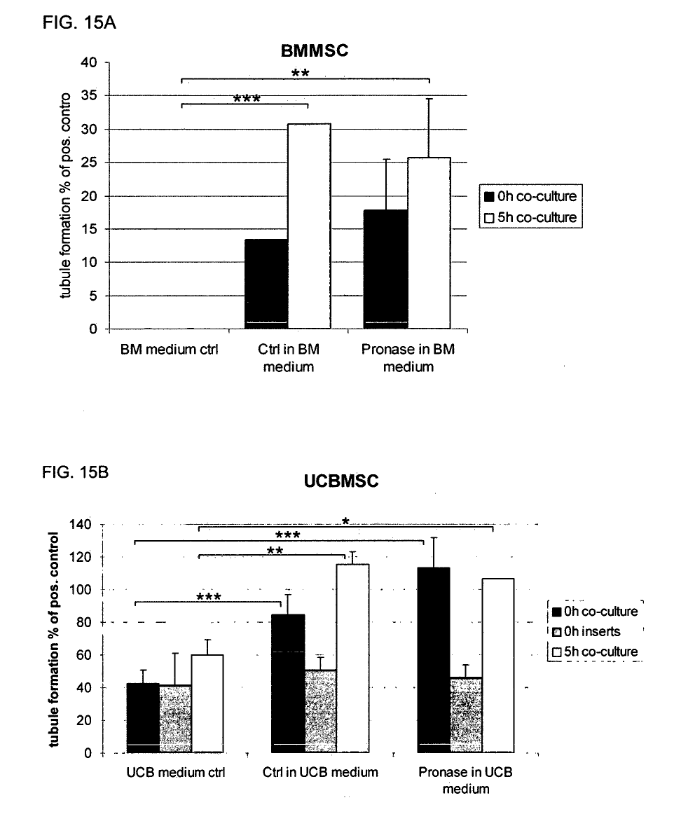

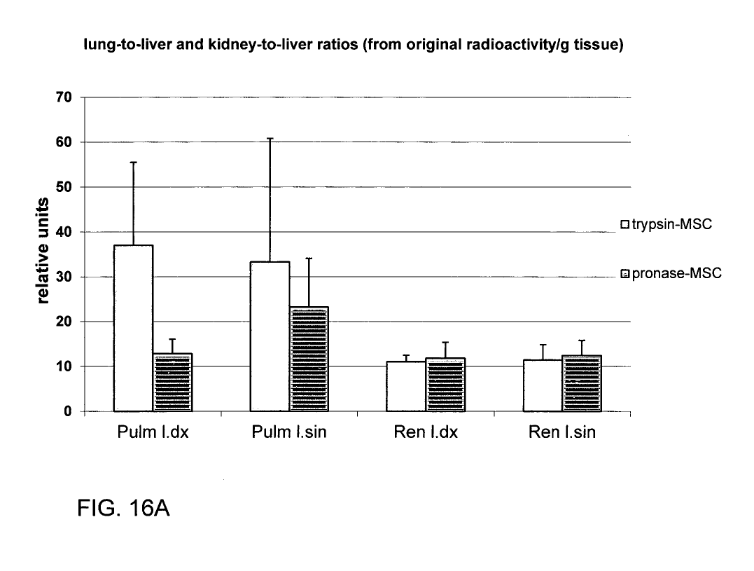

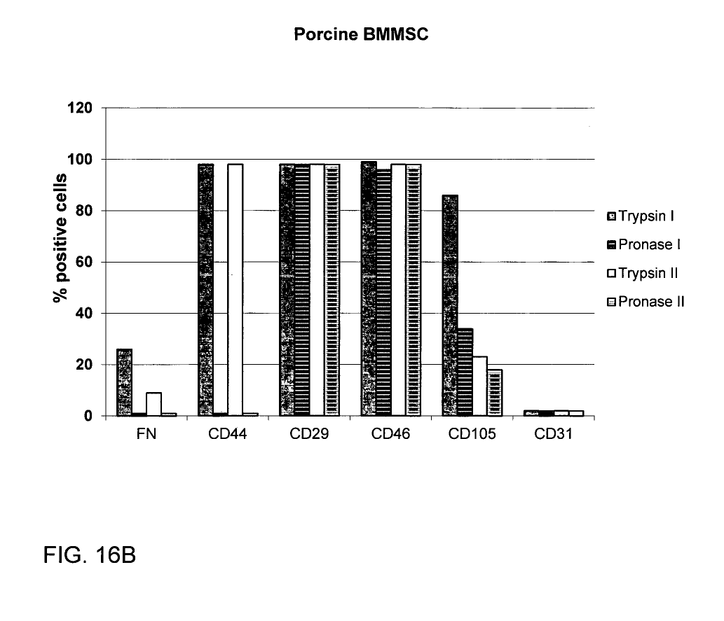

The biopsies from the organs were analysed, again the vast majority of the radioactivity was detected in the lungs. The average lung-to-liver ratio in the control, pigs was 37 (right lung) and 33 (left lung) whereas pigs injected with the pronase-treated cells had a lower means of radioactivity in the lungs: 13 (right lung) and 23 (left lung) (FIG. 16A). However, there was a high variation between the individual pigs. The kidneys had relatively a high relative amount of radioactivity, which was similar in both groups. Other organs, cerebrum, cerebellum, thymus, heart, liver, spleen, kidneys, and mesenterium had only minimal levels of radioactivity.

When porcine MSCs were treated with pronase and analyzed for cell surface expression using flow cytometry, similar changes were seen on the cell surface as in human MSCs. Both fibronectin and CD44 levels were dramatically decreased (FIG. 16B).

### EXAMPLE 13

### Materials and Methods

A sample of pronase enzyme (Roche, #10165921001, lot 70299926) was analysed with mass spectrometry in order to identify the proteins within the sample. The sample was treated overnight in PBS, containing 0.5 mM EDTA and 10% (w/w) modified Trypsin (sequencing grade, Promega Ltd) at 37° C. The protein identification was done both after SDS-PAGE gel separation and using the in-liquid reduction, alkylation and digestion procedure described in Example 8.

SDS-PAGE separation was carried out using 12% gel, which was silver-stained as described (Electrophoresis 1997, 18, 349-359). The lower portion of the gel with proteolytically digested protein pieces was selected for in-gel protein digestion. Here, gel pieces were washed and proteins reduced, alkylated and tryptically digested over night as described (Shevchenko et al (2006) In-gel digestion for mass spectrometric characterization of proteins and proteomes. Nature Protoc 1: 2856-2860. 10.1038/nprot.2006.468).

Digested peptides, either from in-liquid or in-gel digest, were analyzed by mass spectrometry essentially as described in Example 8. Protein identification was performed with Mascot Server (Matrix Science Ltd., version 2.2.07) against proteins of Streptomyces Griseus in UniProt database (release 2011 _{—}03) using search criteria: one potential misscleavage site, variable modifications of carbamidomethyl and propionamide, semitryptic cleavage pattern.

### Results

To characterise typical content of pronase enzyme mixture, an aliquot (Roche, #10165921001, lot 70299926) was analysed with mass spectrometry with two alternative ways. Altogether 16 proteins were found in the direct digestion sample and 38 proteins were found in the gel-digested sample. Seven proteins were found in common between the samples; they were identified with the highest identification scores in both samples. These proteins were: metalloendopeptidase, aminopeptidase, trypsin, putative secreted subtilisin-like serine protease, carboxypeptidase, chain E of structures of product and inhibitor complexes of Streptomyces Griseus protease A, and aminopeptidase S.

1. A method of modifying the a cell surface of a stem cell by treating the cell with a proteolytic enzyme.

2. The method according to claim 1, wherein the enzyme is pronase and/or proteinase K.

3. The method according to claim 1, wherein the stem cell is a mesenchymal stem cell or a hematopoietic stem cell.

4. The method of claim 1 wherein the enzyme is pronase.

5. The method according to claim 1, wherein the enzyme is proteinase K.

6. The method according to claim 1, wherein the stem cell is a mesenchymal stem cell.

7. A stem cell and/or a population thereof having cell surface protein and/or proteoglycan profile, wherein proteins fibronectin and CD44 are essentially missing.

8. The stem cell and/or a population according to claim 7, wherein the level of at least one of the proteins CD49d, CD49e, CD105, galectin-1, CD166, CD146, or CSPG4 is diminished.

9. The stem cell and/or a population according to claim 7, wherein the level of at least one of the proteins CD49d, CD49e, CD105, galectin-1, CD166, CD146, or CSPG4 is essentially missing.

10. The stem cell population according to claim 7, wherein proteins CD90, CD29 and CD13 are present in the profile.

11. The stem cell and/or a population according to claim 7, wherein the profile results from the treatment with a proteolytic enzyme.

12. The stem cell and/or a population according to claim 11, wherein the enzyme treatment is made with pronase and/or proteinase K.

13. The stem cell and/or a population according to claim 7, wherein the stem cell is a mesenchymal stem cell or a hematopoietic stem cell.

14. A method of hindering and/or preventing the transition of stem cells of a graft to an organ that is not the actual target one, wherein the cells are treated with a proteolytic enzyme.

15. The method of claim 14, wherein the organ that is not the actual target one is lungs and/or liver.

16. The method according to claim 14, wherein the proteolytic enzyme is pronase and/or proteinase K.

17. The method of claim 14, wherein the proteolytic enzyme is pronase.

18. The stem cell and/or population of claim 11, wherein the enzyme treatment is made with pronase.

19. The method according to claim 14, wherein the stem cells are mesenchymal stem cells or hematopoietic stem cells.

20. The method of claim 14, wherein the transition of the stem cells of the graft to the target organ of an individual is assisted.

21. The method of claim 14, wherein the distribution behaviour of the stem cells in the graft is modified and/or altered.
